(19) United States
(12) Patent Application Publication
(10) Pub. No.: US 2003/0077249 A1
(43) Pub. Date: Apr. 24, 2003

(54) CELL ACTIVATION PROCESS AND REAGENTS THEREFOR

(76) Inventors: CHRISTOPHER ROBERT BEBBINGTON, BERKSHIRE (GB); ALASTAIR DAVID GRIFFITHS LAWSON, HANIS (GB); ANDREW NEIL, CHARLES WEIR, BERKSHIRE (GB); HELENE MARGARET FINNEY, BERKSHIRE (GB)

Correspondence Address: Dike, Bronstein, Roberts & Cushman, EDWARDS & ANGELL, P.O. Box 9169, Boston, MA, 02209, US

(21) Appl. No.: 09/091,608

(22) PCT Filed: Dec. 23, 1996
(86) PCT No.: PCT/GB96/03209

(51) Int. Cl. A61K 48/00; C12Q 1/68; C07H 21/04; C12P 21/04

(52) U.S. Cl. 424/93.2; 514/44; 536/23.4; 435/6; 435/320.1; 435/325; 435/366; 435/455; 435/69.7; 435/69.1; 424/93.21

(57) ABSTRACT

A cell activation process is described in which an effector cell is transformed with DNA coding for a chimeric receptor containing two or more different cytoplasmic signalling components. The activated cell may be of use in medicine for example in the treatment of diseases such as cancer

This invention relates to a process for activating cells, a DNA delivery system for achieving cell activation and the use of activated cells in medicine.

The natural T-cell receptor is a complex association of polypeptide chains comprising antigen binding, transmembrane and cytoplasmic components. Binding of antigen to the receptor in the correct context triggers a series of intracellular events leading to activation of the T-cell and for example destruction of the antigen presenting target cell. Before recognition of the antigen can take place, the antigen must be presented in association with MHC molecules.

It would be highly desirable if this requirement for MHC could be bypassed by engineering T-cells to become active on binding ligands other than a natural MHC-presented antigen. This would provide a means of avoiding the variability between individuals associated with MHC presentation and would also permit the targeting of more highly expressed surface antigens thereby increasing the efficacy of lymphocyte mediated therapy, for example in tumour therapy.

Chimeric receptors have been designed to target T-cells to cells expressing antigen on their cell surface. Such recombinant chimeric receptors include chimeras containing binding domains from antibodies and intracellular signalling domains from the T-cell receptor, termed ‘T-bodies’ [see for example Published International Patent Specifications Nos. WO 92/10591, WO 92/15322, WO 93/19163 and WO 95/02686].

The recombinant chimeric receptors described in the art are composed of a ligand binding component, a transmembrane component and a cytoplasmic component. It has been found however, that transfection of T-cells with these recombinant chimeric receptors does not result in acceptable levels of T-cell activation upon antigen binding unless the T-cell is also co-stimulated by, for example, treatment with high levels of interleukin 2 [II-2]. The need for co-stimulation makes the method suitable principally for ex-vivo treatment of patients. This is a lengthy and complicated procedure.

The present invention offers an alternative to the present ex-vivo approach in that it achieves improved ex-vivo activation without the need for addition of costimulating agents such as II-2. It also advantageously provides successful in-vivo redirection and activation of T-cells, particularly in response to a single type of extracellular interaction.

Essentially the invention provides an effector cell which has been transformed with DNA coding for a chimeric receptor. The chimeric receptor contains two or more different signalling cytoplasmic components which are not naturally linked and which advantageously are chosen to act together cooperatively to produce improved activation of the cell. DNA coding for such recombinant chimeric receptors may be introduced into T-cells or other effector cells in-vivo and/or ex-vivo. Subsequent binding of an effector cell expressing one or more of these chimeric receptors to a target cell elicits signal transduction leading to activation of the effector cell in a process involving clustering or dimerisation of chimeric receptors or allosteric changes in the chimeric receptor or another mechanism for receptor-triggering.

Thus according to one aspect of the invention we provide a method of activating a cell as a result of one type of extracellular interaction between said first cell and a molecule associated with a second target cell characterised in that said first cell is provided with a DNA delivery system comprising DNA coding for one or more recombinant chimeric receptors comprising two or more different cytoplasmic signalling components, wherein said cytoplasmic components are not naturally linked, and at least one is derived from a membrane spanning polypeptide.

The DNA coding for the chimeric receptor(s) is arranged such that when it is expressed, and on the extracellular interaction between the cell and a second target cell, a signal is transduced via the cytoplasmic signalling components to two or more different intracellular signalling messengers.

This results in activation of the cell and elicits a biological response to the target cell. As used herein, cell activation means activation of one or more signal transduction pathways. This may be evidenced by an increase in cell proliferation; expression of cytokines with, for example pro or anti-inflammatory responses; stimulation of cytolytic activity, differentiation or other effector functions; antibody secretion; phagocytosis; tumour infiltration and/or increased adhesion.

The cytoplasmic signalling components are preferably selected such that they are capable of acting together cooperatively. They are “not naturally linked”, which term is used herein to denote cytoplasmic signalling components which in nature are not connected to each other on a single polypeptide chain. Particularly useful signalling components include those described hereinafter in relation to other aspects of the invention.

In addition to the cytoplasmic signalling components each recombinant chimeric receptor preferably comprises a binding component capable of recognising a cell surface molecule on a target cell, and a transmembrane component. The DNA coding for these components will additionally code for a signal peptide to ensure that the chimeric receptor(s) once expressed will be directed to the cell surface membrane. All the components may be coded for by a single DNA coding sequence.

Alternatively, each cytoplasmic signalling component may be coded for by two or more separate DNA coding sequences. In this instance each DNA coding sequence may also code for a signal peptide, a transmembrane component and/or a binding component. The binding components may be different, but will generally all be capable of participating in the same type of extracellular event, for example by binding to the same molecule associated with the target cell. In one preference the binding components are the same.

In some of the applications described hereinafter, for example where the binding component is an antibody or an antibody fragment, the DNA coding for the chimeric receptor may comprise two separate DNA coding sequences, one sequence for example coding for part of the binding component [in the case of an antibody for example a V_{H} component] linked to the signal peptide, transmembrane and cytoplasmic signalling component(s), and the second sequence coding for the remainder of the binding component [for example a V_{L} compoonent in the example given].

In order to activate a desired cell the DNA coding for the chimeric receptor will first need to be delivered to the cell. Thus according to a second aspect of the invention we provide a DNA delivery system comprising DNA in association with a carrier said DNA coding for a recombinant chimeric receptor capable of one type of extracellular interaction and comprising two or more different cytoplasmic signalling components which are not naturally linked, and wherein at least one of said cytoplasmic components is derived from a membrane spanning polypeptide.

In this aspect of the invention the chimeric receptor may be coded for by a single DNA coding sequence, coding in particular for the two or more different cytoplasmic signalling components. Thus in one preference the invention provides a DNA delivery system comprising DNA in association with a carrier said DNA coding for a recombinant chimeric receptor wherein said DNA codes in reading frame for:

i) a signal peptide component;

ii) a binding component capable of recognising a cell surface molecule on a target cell;

iii) a transmembrane component;

iv) two or more different cytoplasmic signalling components which are not naturally linked, and wherein at least one of said cytoplasmic components is derived from a membrane spanning polypeptide, and optionally

v) one or more spacer regions linking any two or more of said i) to iv) components.

The components of the recombinant chimeric receptor are operatively linked such that the signalling cytoplasmic components are functional in transducing a signal resulting in activation of one or more messenger systems as a result of recognition of a cell surface molecule on a target cell by the binding component.

Two or more of the components may be linked by one or more spacer regions. The spacer regions may function to facilitate the components adopting the correct conformation for biological activity. The use of a spacer region to link the transmembrane component iii) and the binding component ii) is particularly advantageous.

The spacer regions may for example comprise up to 300 amino acids and preferably 20 to 100 amino acids and most preferably 25 to 50 amino acids.

Spacers may be derived from all or part of naturally occurring molecules such as from all or part of the extracellular region of CD8, CD4 or CD28; or all or part of an antibody constant region, including the hinge region. All or part of natural spacing components between functional parts of intracellular signalling molecules for example spacers between ITAMS (immunoreceptor tyrosine based activation motifs) may also be used. Alternatively the spacer may be a non-naturally occurring sequence.

The binding component ii) may be any molecule capable of interacting with cell surface molecules and may be chosen to recognise a surface marker expressed on cells associated with a disease state such as for example those associated with virally infected cells; bacterially infected cells; cancer cells, such as the bombesin receptor expressed on lung tumour cells, carcinoembryonic antigen, polymorphic epithelial mucin, and CD33; peptide hormones, adhesion molecules, inflammatory cells present in autoimmune disease, or a T-cell receptor or antigen giving rise to autoimmunity.

Suitable binding components for use in the chimeric receptors of the invention also include all or part of receptors associated with binding to cell surface associated molecules: the T-cell receptor; CD4; CD8: CD28; cytokine receptors e.g. an interleukin receptor, TNF receptor, or interferon receptor e.g. γ-IFN: receptors for colony stimulating factors e.g. GMCSF; antibodies and antigen binding fragments thereof including for example Fab, Fab′, F(ab′)₂, single chain Fv, Fv, and V_{H} or V_{L} components which may be in association with C_{H} and C_{L} domains. The antibodies or fragments may be murine, human, chimeric or engineered human antibodies and fragments. As used herein the term engineered human antibody or fragment is intended to mean an antibody or fragment which has one or more CDR's and one or more framework residues derived from one antibody, e.g. a murine antibody embedded in an otherwise human framework. Such antibodies are well known and may be prepared by a number of methods for example as described in International Patent Specification No. WO91/09967.

Particularly useful binding components include Fab′ fragments or, especially, single chain Fv fragments.

When the binding component is an antibody or antibody fragment other than a single chain Fv or V_{H} or V_{L} component which contains separate binding chains it will be necessary to include a second separate DNA coding sequence in the delivery system according to the invention to code for the second binding chain. In this instance the first DNA sequence containing the cytoplasmic signalling components and one chain of the antibody or fragment will be coexpressed with the second DNA sequence coding for a signal peptide and the second chain of the antibody or fragment so that assembly of the antibody binding component can occur.

Transmembrane components iii) may be derived from a wide variety of sources such as all or part of the alpha, beta or zeta chain of the T-cell receptor, CD28, CD8, CD4, a cytokine receptor, e.g. an interleukin receptor, TNF receptor, or interferon receptor, or a colony stimulating factor receptor e.g. GMCSF.

The binding and transmembrane components may be linked directly or, preferably, by a spacer region. The spacer region may be one or more of the regions described above. Where more than one region is present, for example two regions, these are preferably different regions, for example an antibody hinge region linked to all or part of the extracellular region of CD28.

The spacer and transmembrane components are advantageously chosen such that they have free thiol groups thereby providing the chimeric receptor with multimerisation, particularly dimerisation capacity. Receptors of this type, especially dimers, are particularly preferred and include those which have CD28 components, the zeta chain of the natural T-cell receptor, and/or antibody hinge sequences.

The transmembrane component may or may not be naturally linked to the cytoplasmic component to which it is attached either directly or by means of a spacer.

The cytoplasmic signalling components iv) can for example transduce a signal which results in activation of one or more intracellular messenger systems. It is preferred that each of the cytoplasmic components activates a different messenger system. The intracellular messenger systems which may be activated either directly or indirectly include, for example, one or more kinase pathways such as those involving tyrosine kinase, PKC or MAP kinase; G-protein or phospholipase mediated pathways; calcium mediated pathways; and pathways involving synthesis of a cytokine such as an interleukin e.g. IL-2, including NFAT, and cAMP mediated pathways.

Examples of suitable cytoplasmic components iv) include, for example those derived from the T-cell receptor such as all or part of the zeta, eta or epsilon chain; CD28; the γ chain of a Fc receptor; or signalling components from a cytokine receptor e.g. interleukin, TNF and interferon receptors, a colony stimulating factor receptor e.g. GMCSF, a tyrosine kinase e.g. ZAP-70, fyn, lyk, Itk and syk; an adhesion molecule e.g. LFA-1 and LFA-2, B29, MB-1, CD3 delta, CD3 gamma, CD5 or CD2. The signalling cytoplasmic components are preferably ITAM containing cytoplasmic components

The cytoplasmic signalling components are preferably selected so that they act cooperatively. They may be in any orientation relative to one another. Particularly useful components include all or part of the signalling component of CD28 or the zeta chain of the T-cell receptor.

The signal component may be that naturally associated with the binding component or may be derived from other sources.

Examples of suitable signal peptide components i) include immunoglobulin signal sequences.

The signal component, binding component, transmembrane component, and cytoplasmic components are preferably derived from or based on human sequences.

Homologues of the individual components of the chimeric receptor may be used and the invention is to be understood to extend to such use. The term homologue as used herein with respect to a particular nucleotide or amino acid sequence coding for a component of the chimeric receptor represents a corresponding sequence in which one or more nucleotides or amino acids have been added, deleted, substituted or otherwise chemically modified provided always that the homologue retains substantially the same function as the particular component of the chimeric receptor. Homologues may be obtained by standard molecular biology and/or chemistry techniques e.g. by cDNA or gene cloning, or by use of oligonucleotide directed mutagenesis or oligonucleotide directed synthesis techniques or enzymatic cleavage or enzymatic filling in of gapped oligonucleotides.

Fragments of the individual components may also be used wherein one or more nucleotides has been deleted provided that the fragment retains substantially the same function as the starting component of the chimeric receptor.

The DNA for use in this and other aspects of the invention may be obtained from readily available DNA sources using standard molecular biology and/or chemistry procedures, for example by use of oligonucleotide directed mutagenesis or oligonucleotide directed synthesis techniques, enzymatic cleavage or enzymatic filling in of gapped oligonucleotides. Such techniques are described by Maniatis et al in Molecular Cloning, Cold Spring Harbor Laboratory, New York 1989, and in particular in the Examples hereinafter.

The carrier for use in the DNA delivery systems according to the invention may be a vector or other carrier suitable for introduction of the DNA ex-vivo or in-vivo into target cells and/or target host cells. Examples of suitable vectors include viral vectors such as retroviruses, adenoviruses, adenoassociated viruses, EBV, and HSV, and non-viral vectors, such as liposomal vectors and vectors based on DNA condensing agents. Alternatively the carrier may be an antibody. Where appropriate, the vector may additionally include promoter/regulatory sequences and/or replication functions from viruses such as retrovirus LTRs, AAV repeats, SV40 and hCMV promoters and/or enhancers, splicing and polyadenylation signals; EBV and BK virus replication functions. Tissue specific regulatory sequences such as the TCR-α promoter, E-selectin promoter and the CD2 promoter and locus control region may also be used.

Where two or more DNA molecules are used in the DNA delivery system they may be incorporated into the same or different carriers as described above.

For ex-vivo use, the DNA delivery system of the invention may be introduced into effector cells removed from the target host using methods well known in the art e.g. transfection, transduction, biolistics, protoplast fusion, calcium phosphate precipitated DNA transformation, electroporation, cationic lipofection, or targeted liposomes. The effector cells are then reintroduced into the host using standard techniques.

A wide variety of target hosts may be employed according to the present invention such as, for example, mammals and, especially, humans.

Examples of suitable effector cells include cells associated with the immune system such as lymphocytes e.g. cytotoxic T-lymphocytes, tumour infiltrating lymphocytes, natural killer cells, neutrophils, basophils or T-helper cells; dendritic cells, B-cells, haemoatopaietic stem cells, macrophages, monocytes or NK cells. The use of cytotoxic T-lymphocytes is especially preferred.

The DNA delivery system according to the invention is particularly suitable for in vivo administration. It may be in one preferred example in the form of a targeted delivery system in which the carrier is capable of directing the DNA to a desired effector cell. Particular examples of such targeted delivery systems include targeted-naked DNA, targeted liposomes encapsulating and/or complexed with the DNA, targeted retroviral systems and targeted condensed DNA such as protamine and polylysine condensed DNA.

Targeting systems are well known in the art and include using, for example, antibodies or fragments thereof against cell surface antigens expressed on target cells in vivo such as CD8; CD16; CD4; CD3; selectins e.g. E-selectin; CD5; CD7; CD34; activation antigens e.g. CD69 and IL-2R. Alternatively, other receptor-ligand interactions can be used for targeting e.g. CD4 to target HIV_{gp}160-expressing target cells.

In general the use of antibody targeted DNA is preferred, particularly antibody targeted naked DNA, antibody targeted condensed DNA and especially antibody targeted liposomes. Particular types of liposomes which may be used include for example pH-sensitive liposomes where linkers cleaved at low pH may be used to link the antibody to the liposome. Cationic liposomes which fuse with the cell membrane and deliver the recombinant chimeric receptor DNA according to the invention directly into the cytoplasm may also be used. Liposomes for use in the invention may also have hydrophilic groups attached to their surface to increase their circulating half-life such as for example polyethylene glycol polymers. There are many examples in the art of suitable groups for attaching to liposomes or other carriers; see for example International Patent 91/05546, WO 93/19738, WO 94/20073 and WO 94/22429. The antibody or other targeting molecule may be linked to the DNA, condensed DNA or liposome using conventional readily available linking groups and reactive functional groups in the antibody e.g. thiols, or amines and the like, and in the DNA or DNA containing materials.

Non-targeted delivery systems may also be used and in these targeted expression of the DNA is advantageous. Targeted expression of the DNA may be achieved for example by using T-cell specific promoter systems such as the zeta promoter and CD2 promoter and locus control region, and the perforin promoter.

The aspect of the invention described above advantageously utilises a single DNA sequence to code for the chimeric receptor. It will be appreciated however that the invention may be extended to DNA delivery systems in which the chimeric receptor is coded for by two or more separate DNA coding sequences. Thus in one example, a first and second separate DNA coding sequence may be present in the delivery system each of which codes for components i) to iv) and optionally v) in the same reading frame as described above but which differ from each other in that the cytoplasmic signalling component iv) is not the same. The two DNA coding sequences may each code for more than one signalling component providing that at least one component on the first DNA is different to any other signalling component on the second DNA. As above, the signalling components are advantageously selected to act cooperatively and the remaining components may be any of those previously described for the single DNA embodiment. The binding component iv) coded for by the first DNA will preferably be the same as that coded for by the second DNA. Advantageously the binding component coded by the first DNA will be separated from the transmembrane component by a different spacer region to that coded by the second DNA.

The delivery system may be used ex vivo and in a further aspect the invention provides effector cells transfected with a DNA delivery system according to the invention. The effector cells may be any of those previously described above which are suitable for ex vivo use and are preferably T-cells most preferably cytotoxic T-cells.

The DNA delivery system may take the form of a pharmaceutical composition. It may be a therapeutic or diagnostic composition and may take any suitable form suitable for administration. Preferably it will be in a form suitable for parenteral administration e.g. by injection or infusion, for example by bolus injection or continuous infusion. Where the composition is for injection or infusion, it may take the form of a suspension, solution or emulsion in an oily or aqueous vehicle and it may contain formulatory agents such as suspending, preservative, stabilising and/or dispersing agents. Alternatively, the composition may be in dry form, for reconstitution before use with an appropriate sterile liquid.

If the composition is suitable for oral administration the formulation may contain, in addition to the active ingredient, additives such as: starch—e.g. potato, maize or wheat starch or cellulose—or starch derivatives such as microcrystalline cellulose; silica; various sugars such as lactose; magnesium carbonate and/or calcium phosphate. It is desirable that, if the formulation is for oral administration it will be well tolerated by the patient's digestive system. To this end, it may be desirable to include in the formulation mucus formers and resins. It may also be desirable to improve tolerance by formulating the compositions in a capsule which is insoluble in the gastric juices. It may also be preferable to include the composition in a controlled release formulation.

The DNA delivery system according to the invention is of use in medicine and the invention extends to a method of treatment of a human or animal subject, the method comprising administering to the subject an effective amount of a DNA delivery system described above. The exact amount to be used will depend on the ages and condition of the patient, the nature of the disease or disorder and the route of administration, but may be determined using conventional means, for example by extrapolation of animal experiment derived data. In particular, for ex vivo use the number of transfected effector cells required may be established by ex vivo transfection and re-introduction into an animal model of a range of effector cell numbers. Similarly the quantity of DNA required for in vivo use may be established in animals using a range of DNA concentrations.

The DNA delivery system according to the invention may be useful in the treatment of a number of diseases or disorders. Such diseases or disorders may include those described under the general headings of infectious diseases, e.g. HIV infection; inflammatory disease/autoimmunity e.g. rheumatoid arthritis, osteoarthritis, inflammatory bowel disease; cancer; allergic/atopic diseases e.g. asthma, eczema; congenital e.g. cystic fibrosis, sickle cell anaemia; dermatologic, e.g. psoriasis; neurologic, e.g. multiple sclerosis; transplants e.g. organ transplant rejection, graft-versus-host disease; metabolic/idiopathic disease e.g. diabetes.

DNA coding for a chimeric receptor as described herein also forms a feature of the invention, particularly for use in a delivery system described herein.

The invention is further illustrated in the following non-limiting Examples and Figures in which:

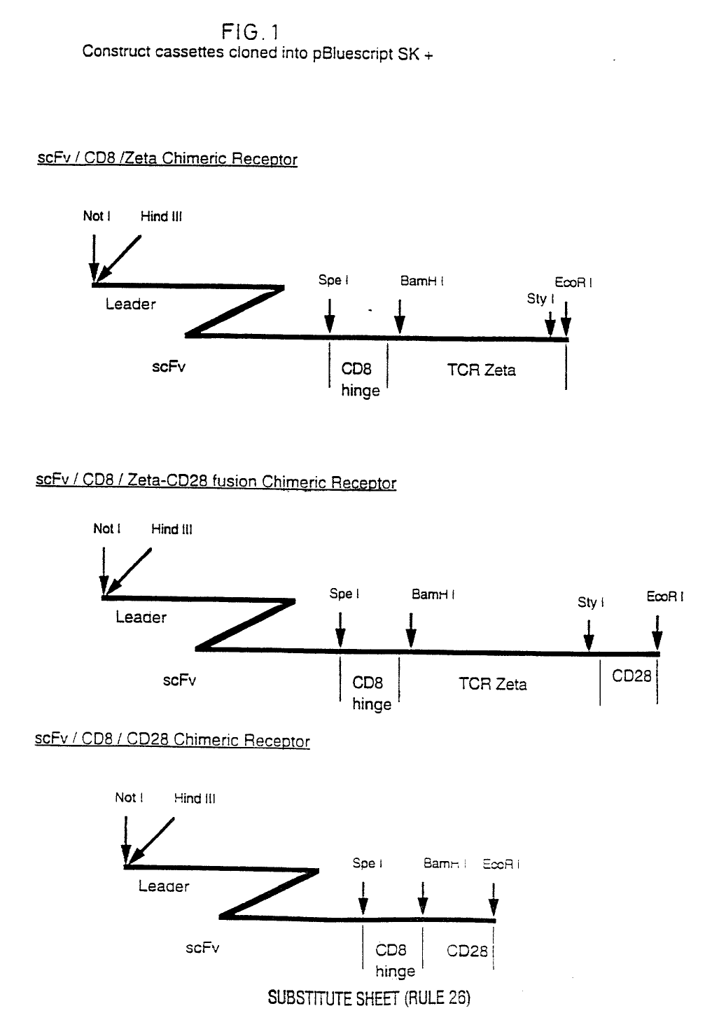

FIG. 1 shows: diagrammatic representation of recombinant chimeric receptor constructs cloned into pBluescript SK+

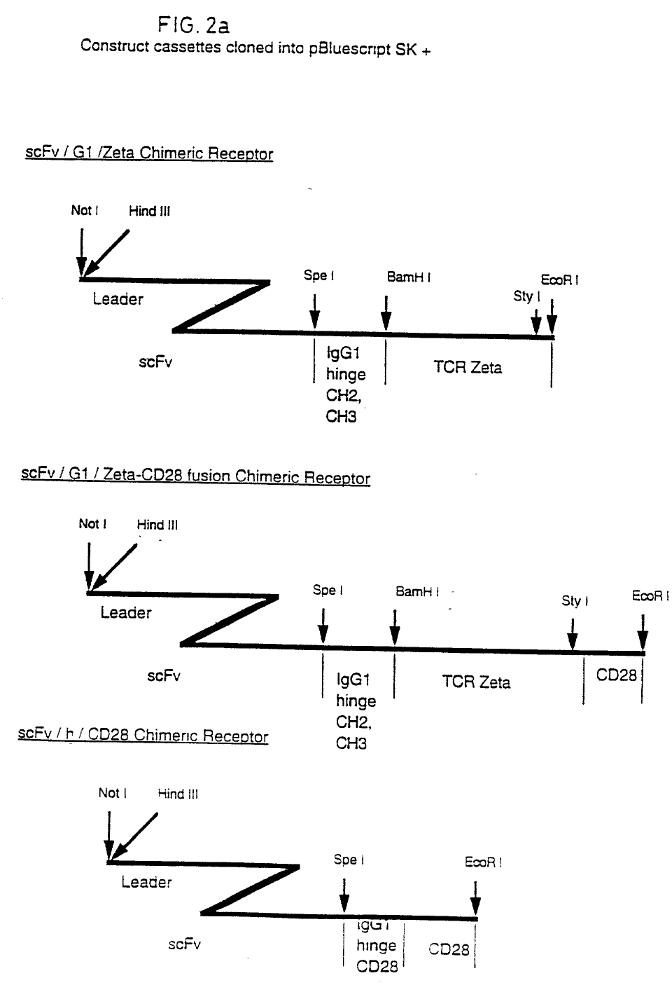

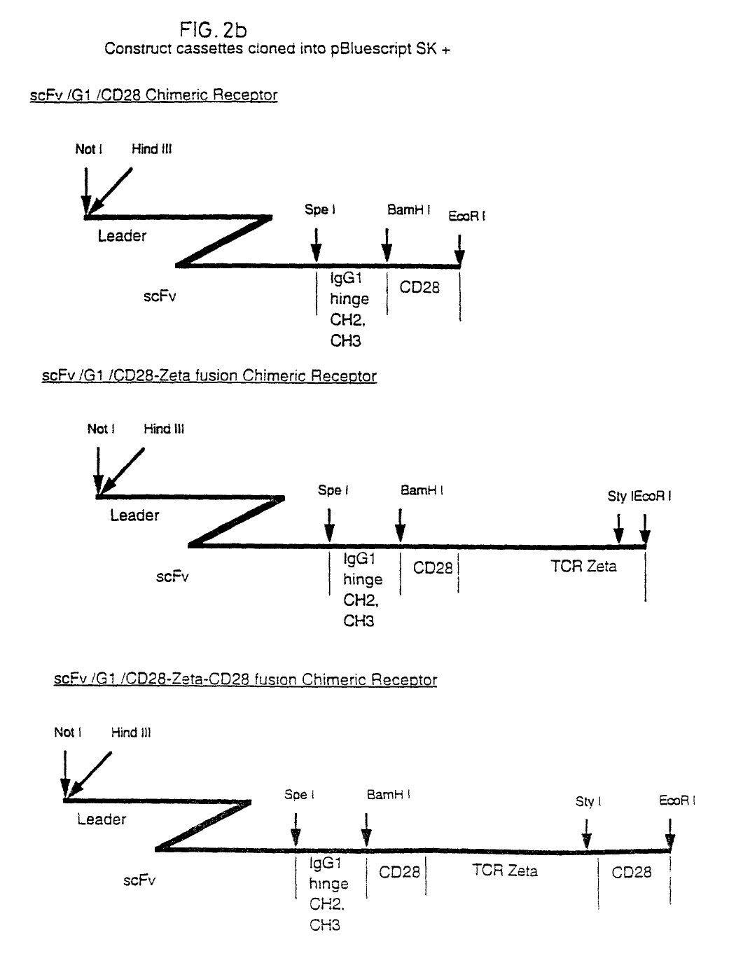

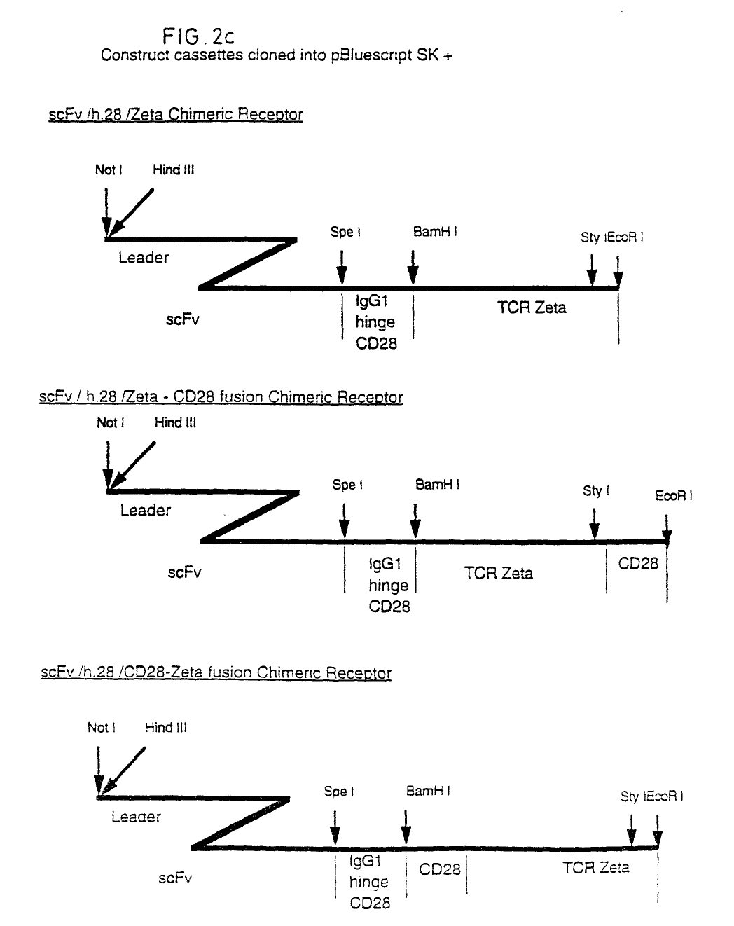

FIG. 2 shows: diagrammatic representation of recombinant chimeric receptor constructs cloned into pBluescript SK+

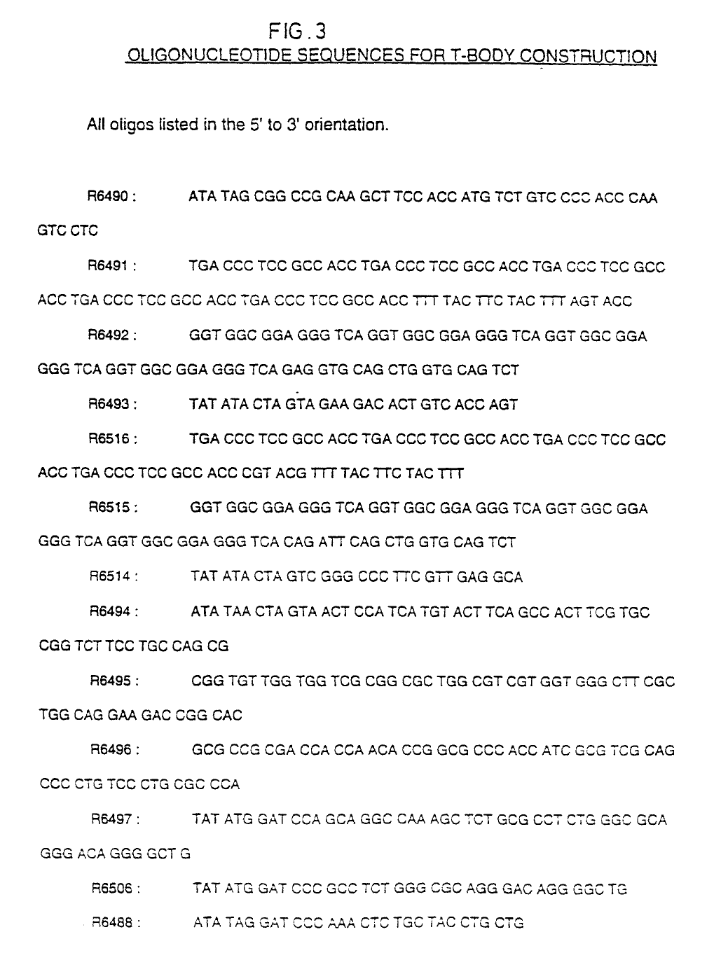

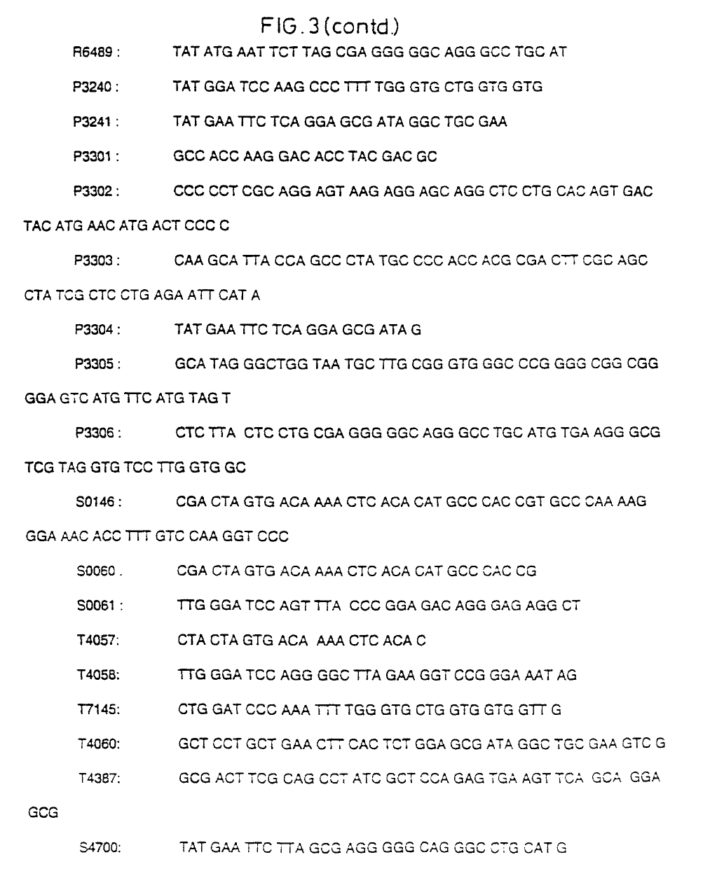

FIG. 3 shows: oligonucleotide sequences for recombinant chimeric receptor construction

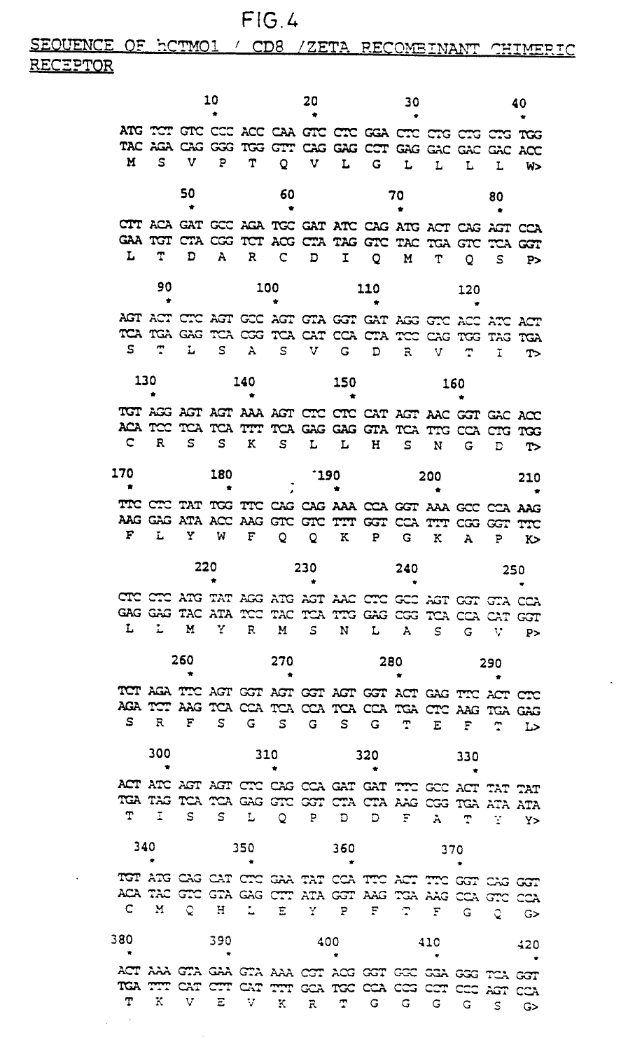

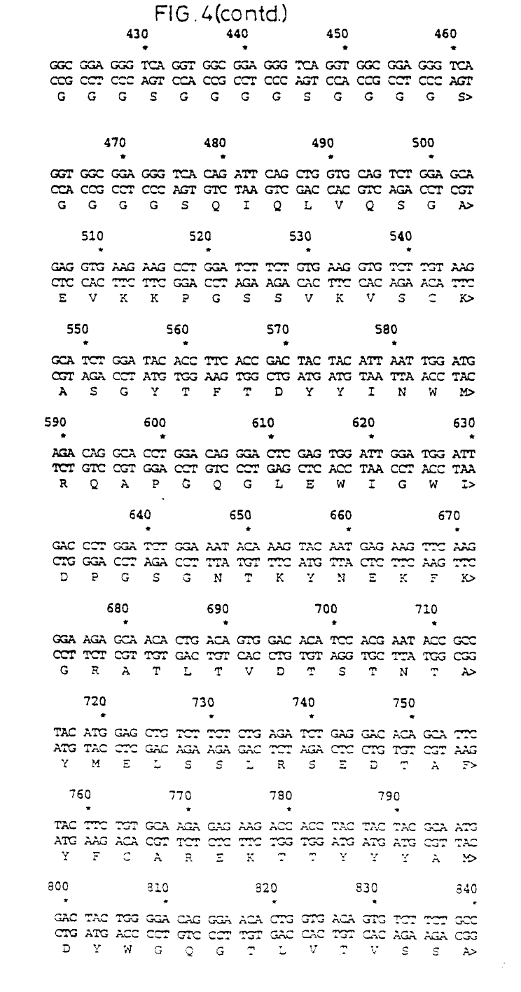

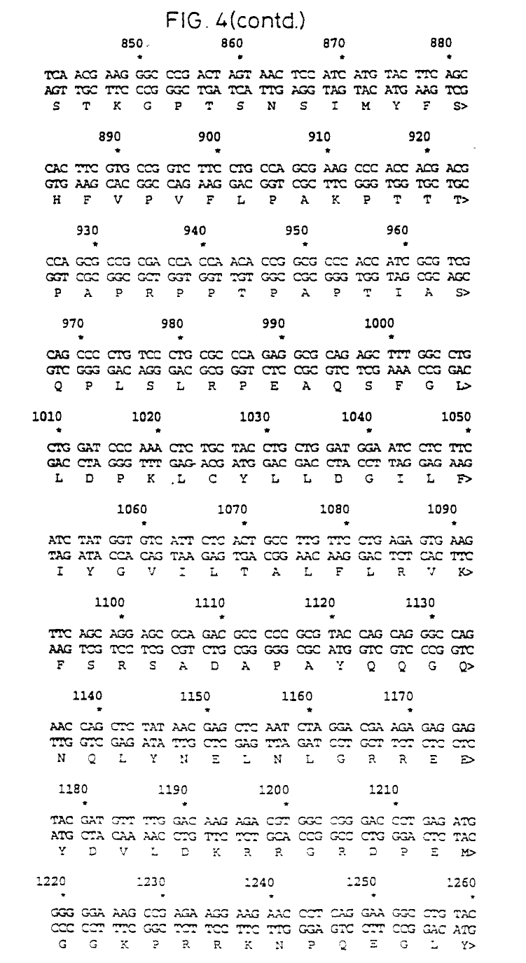

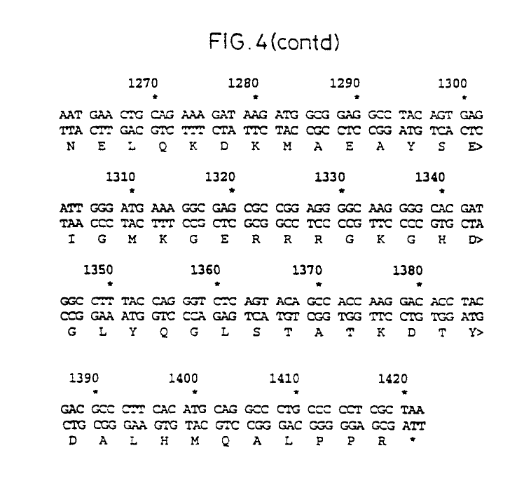

FIG. 4 shows: nucleotide and amino acid sequence of an hCTMO1/CD8/zeta recombinant chimeric receptor

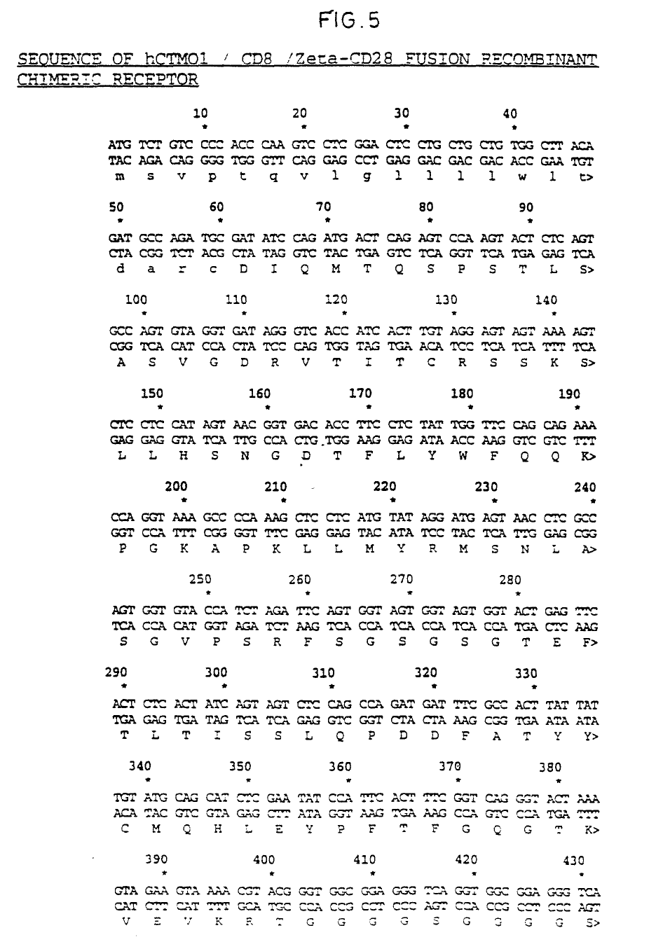

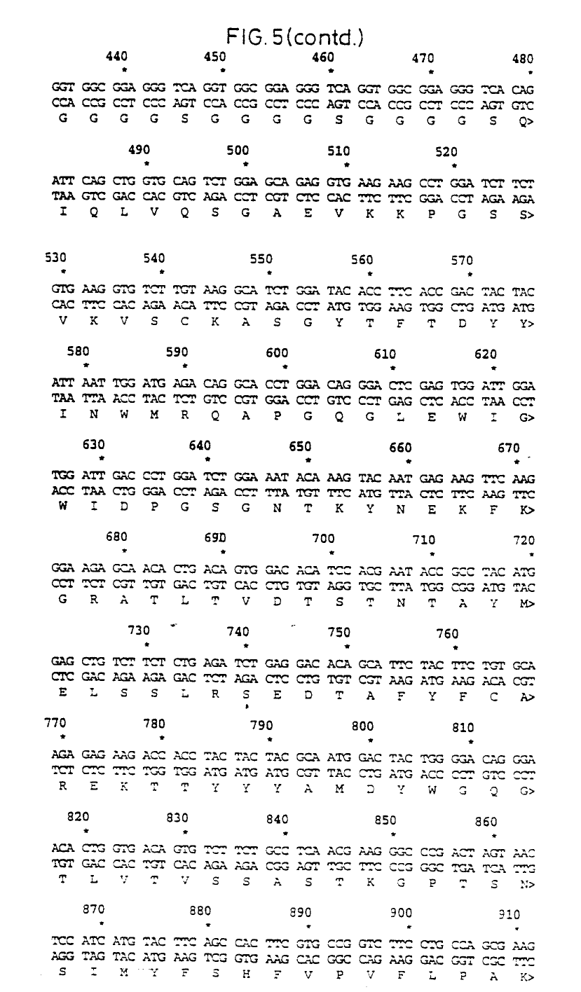

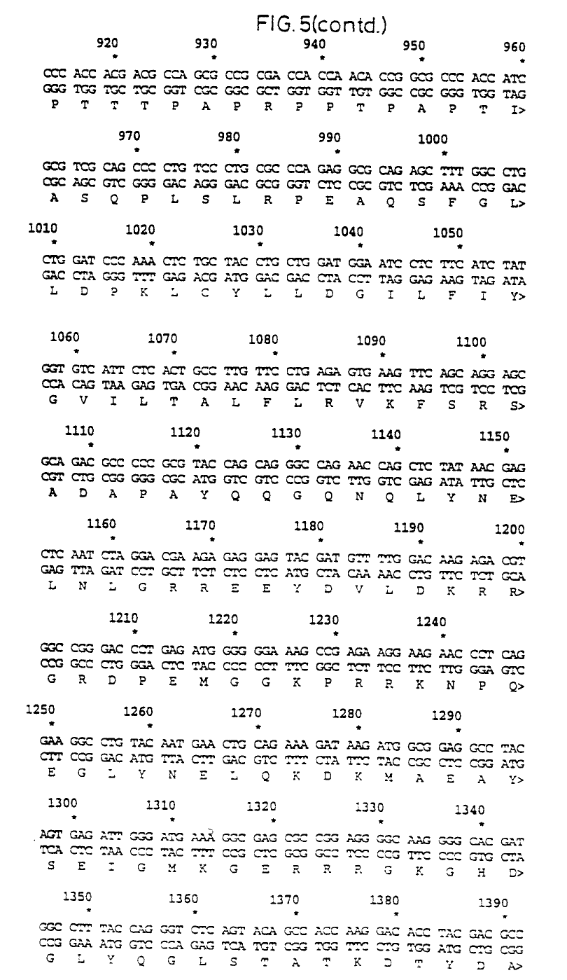

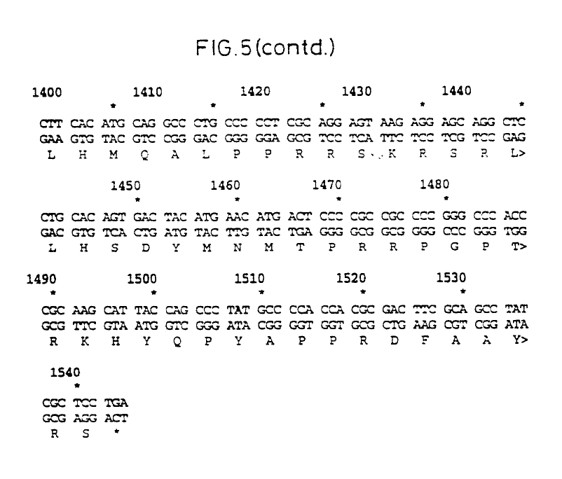

FIG. 5 shows: nucleotide and amino acid sequence of an hCTMO1/CD8/zeta-CD28 recombinant chimeric receptor fusion

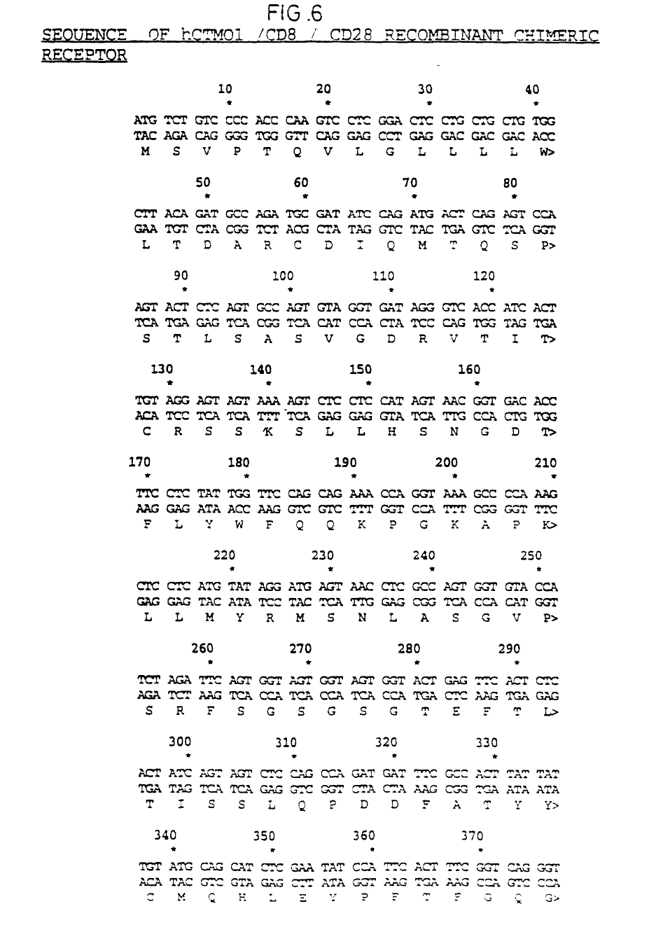

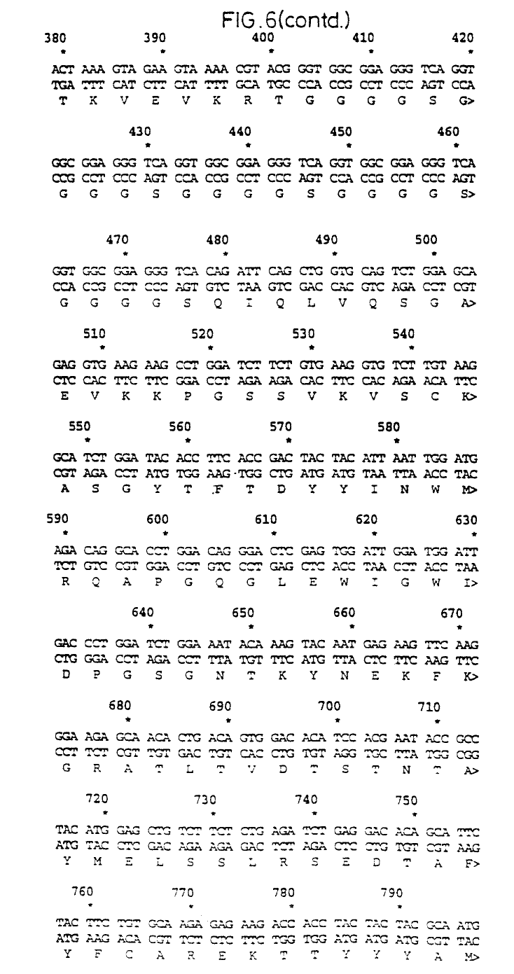

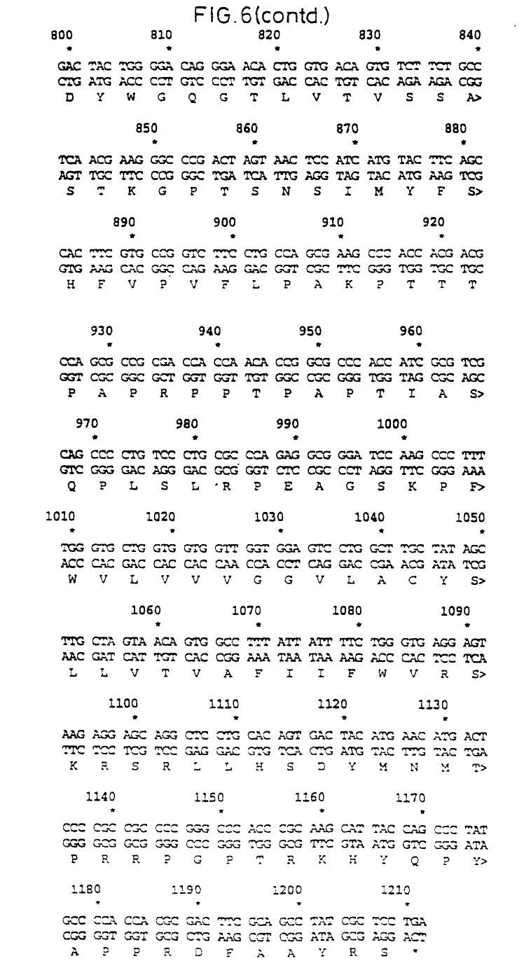

FIG. 6 shows: nucleotide and amino acid sequence of an hCTMO1/CD8/CD28 recombinant chimeric receptor

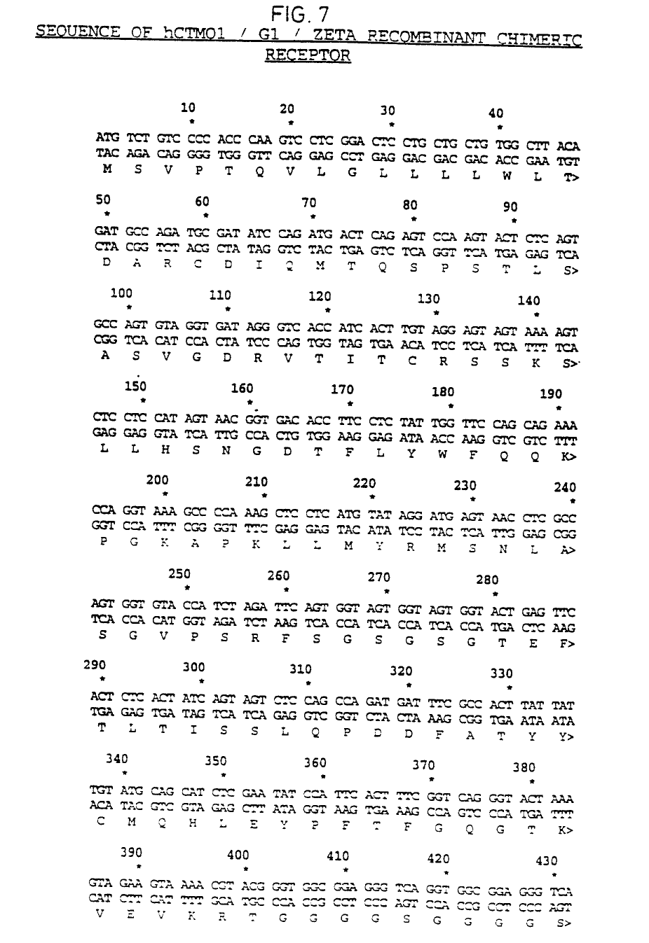

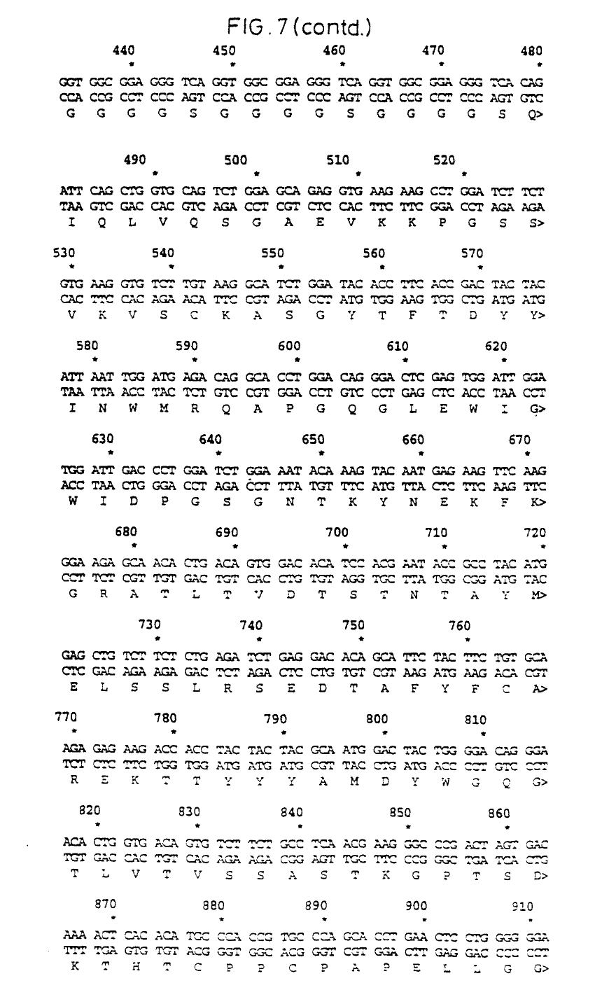

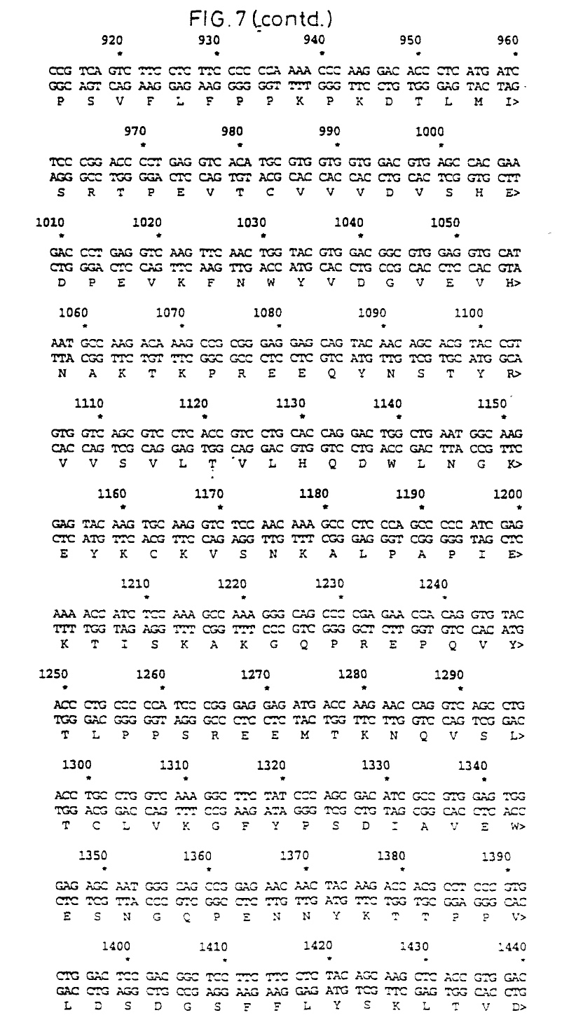

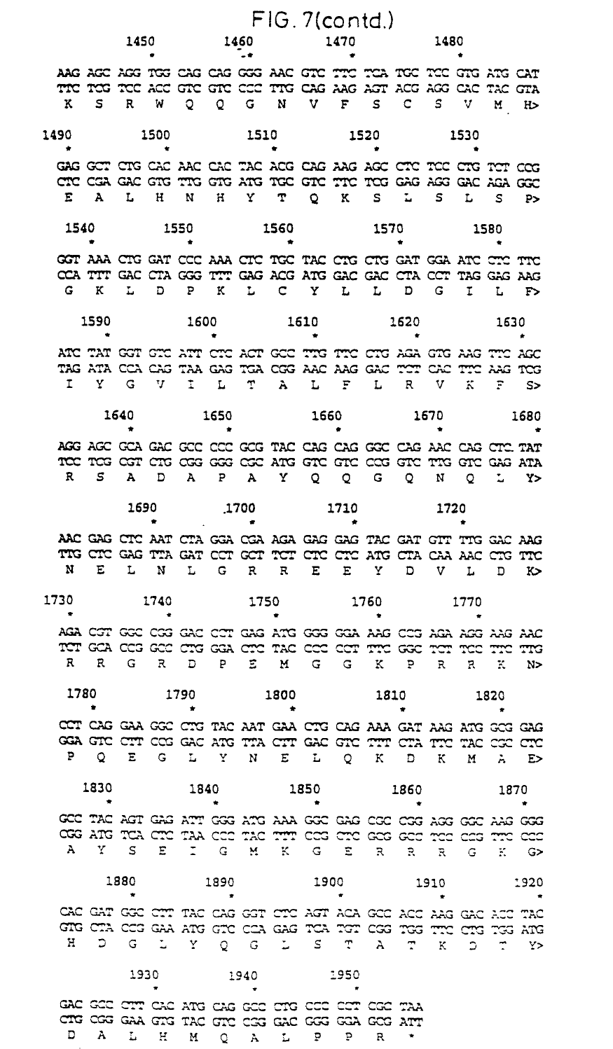

FIG. 7 shows: nucleotide and amino acid sequence of an CTMO1/G1/zeta recombinant chimeric receptor

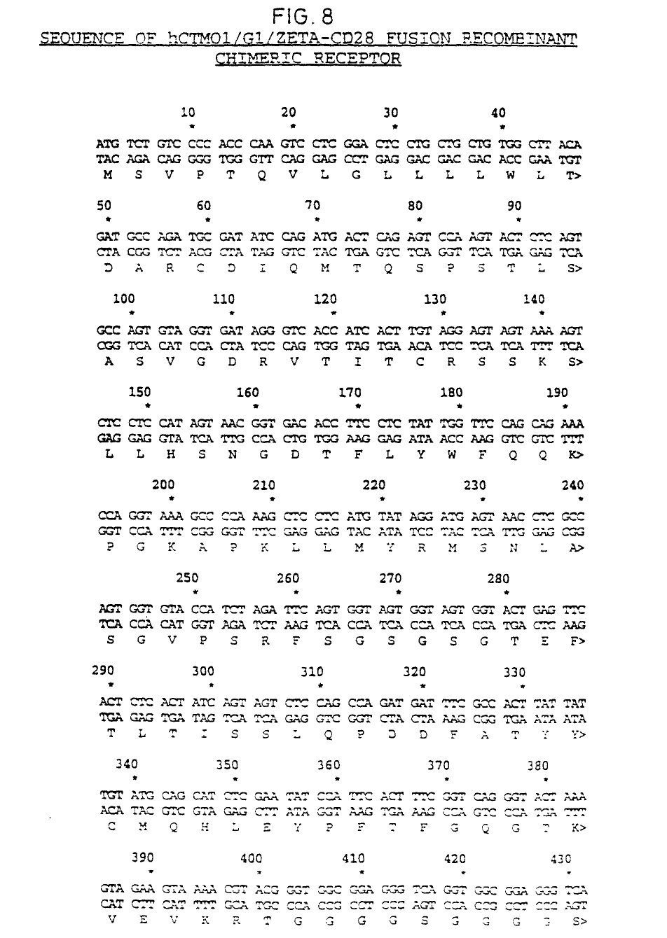

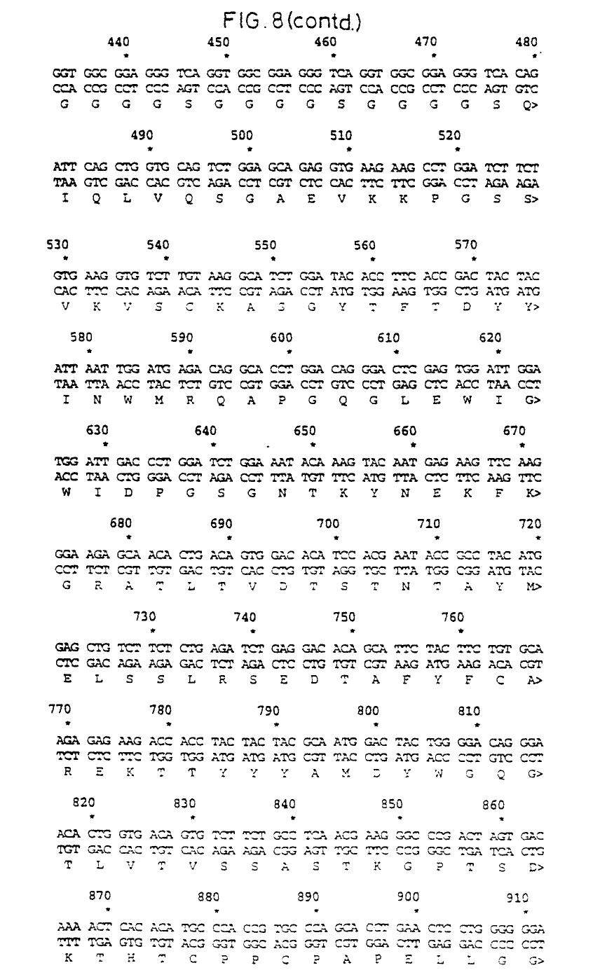

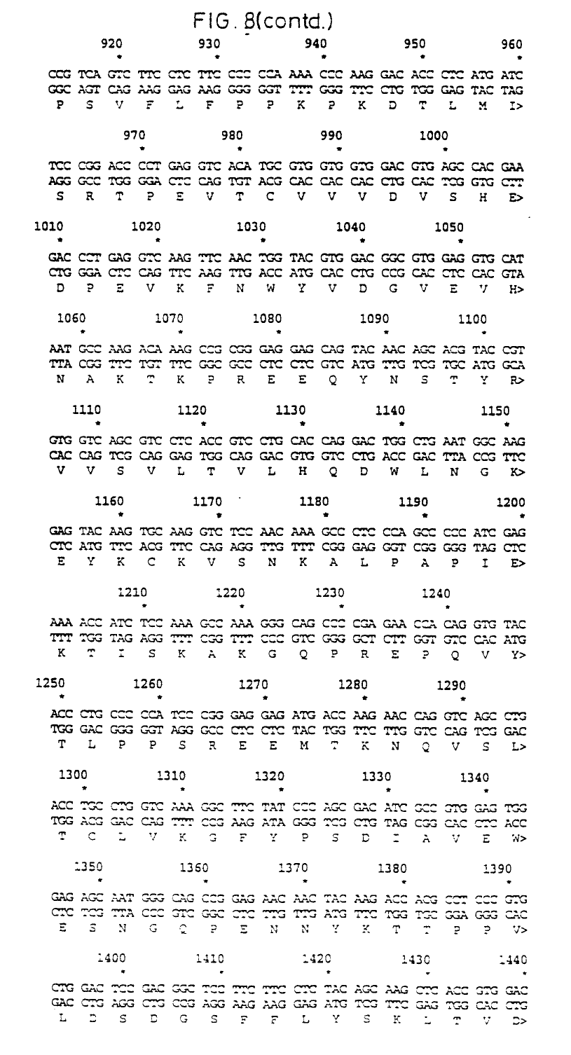

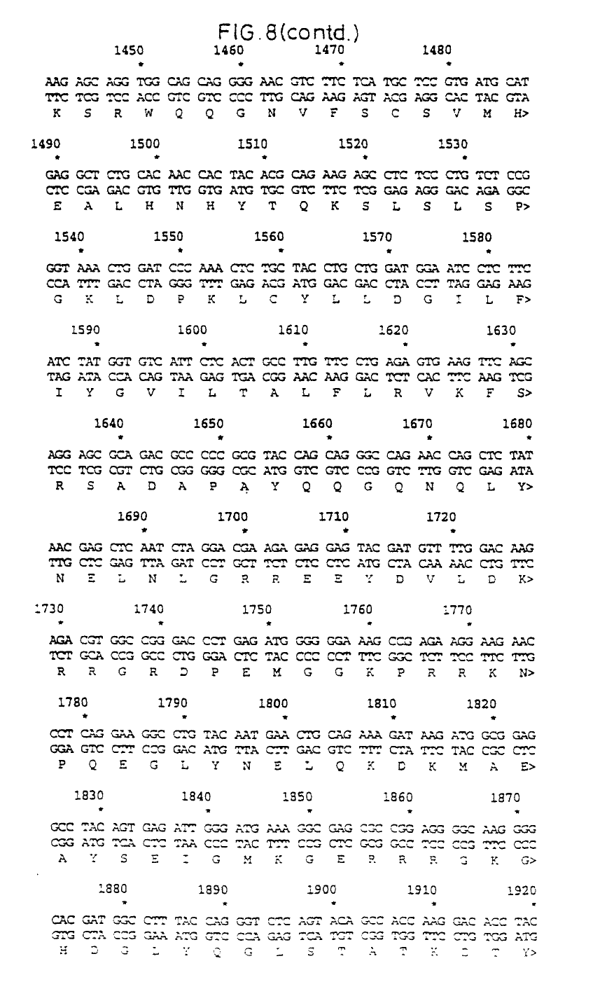

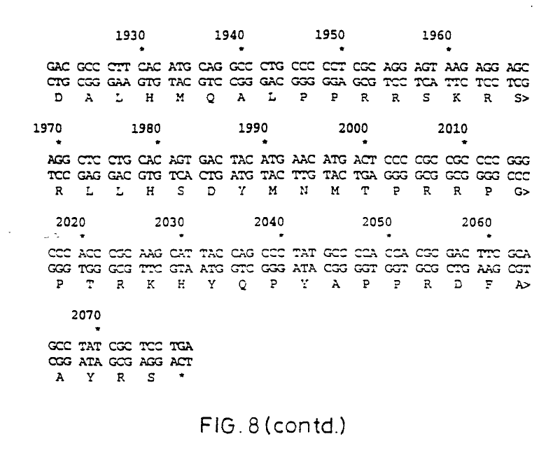

FIG. 8 shows: nucleotide and amino acid sequence of an hCTMO1/G1/zeta-CD28 recombinant chimeric receptor fusion

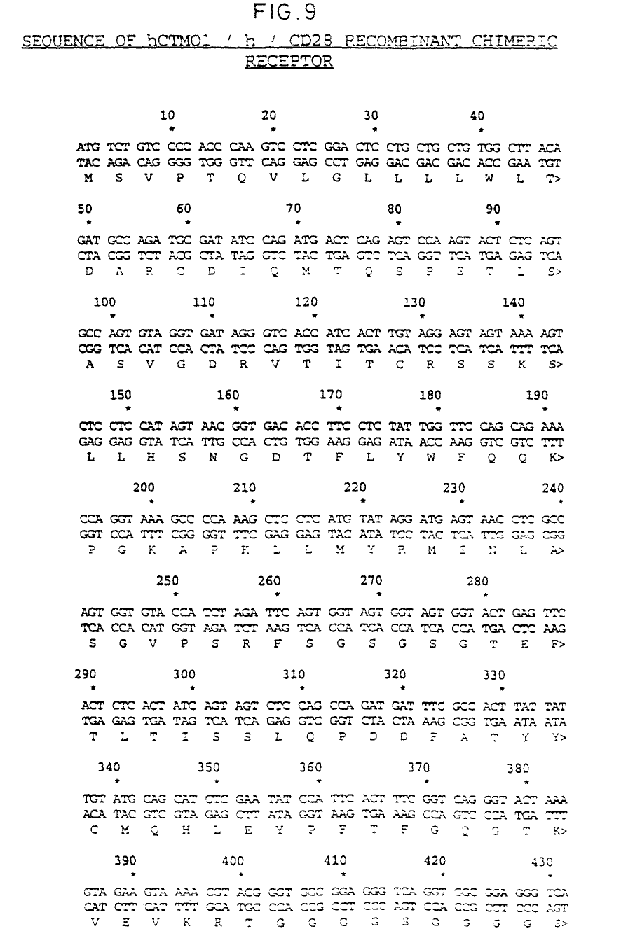

FIG. 9 shows: nucleotide and amino acid sequence of an hCTMO1/h/CD28 recombinant chimeric receptor

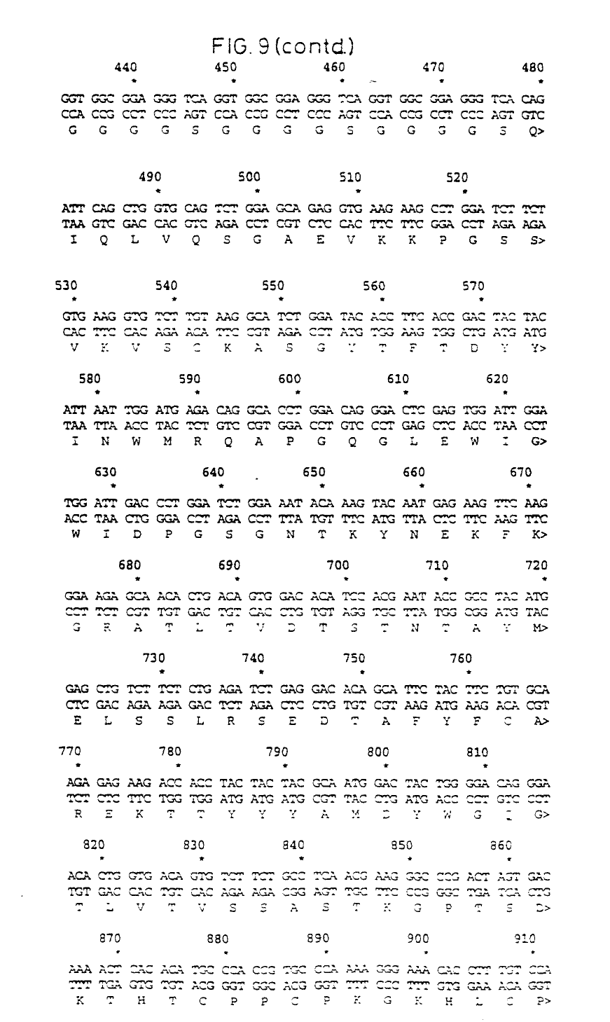

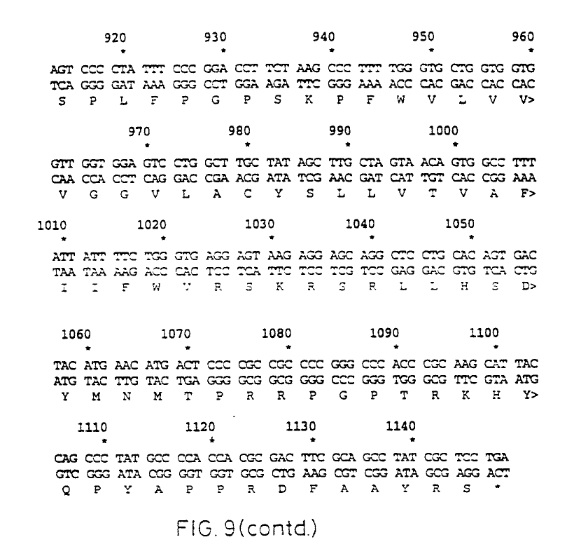

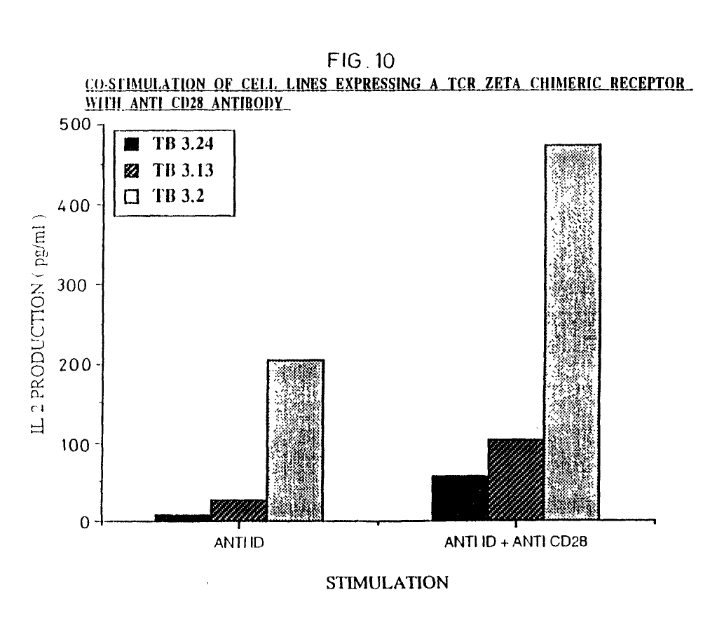

FIG. 10 shows: histogram representation of IL2 production by cell lines TB3.2, 3.13 and 3.24 when stimulated with an anti-idiotypic antibody alone or in combination with an anti-CD28 antibody

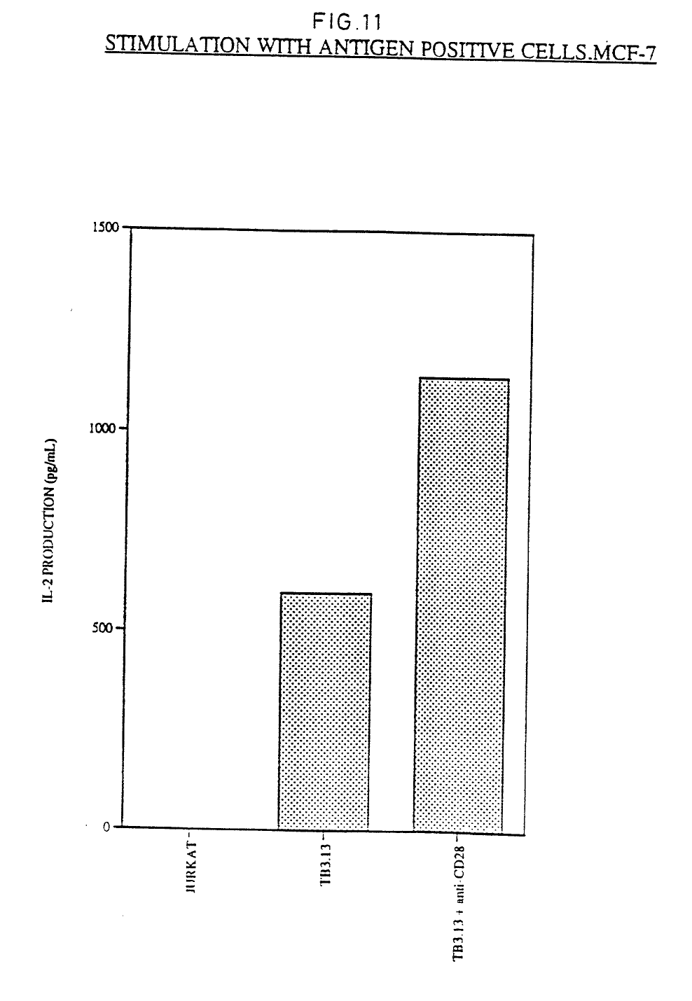

FIG. 11 shows: histogram representation of the production of IL2 by cell line TB3.13 when stimulated with antigen expressing tumour cells, shown with and without co-stimulation using an anti-CD28 antibody.

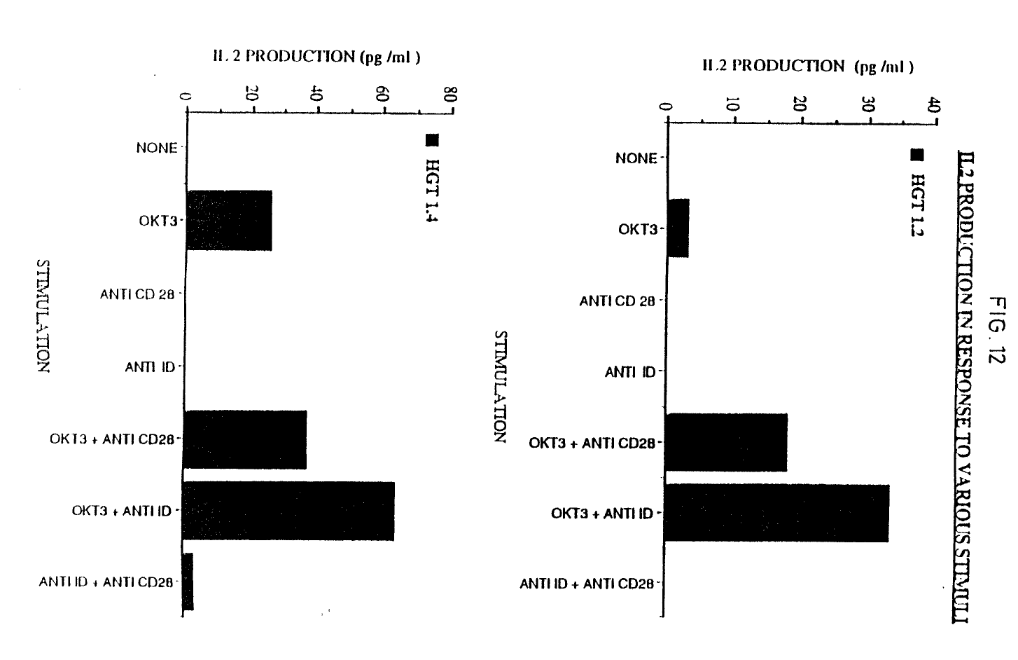

FIG. 12 shows: histogram representation of IL-2 production by HGT1.2 and HGT1.4 in response to various stimuli

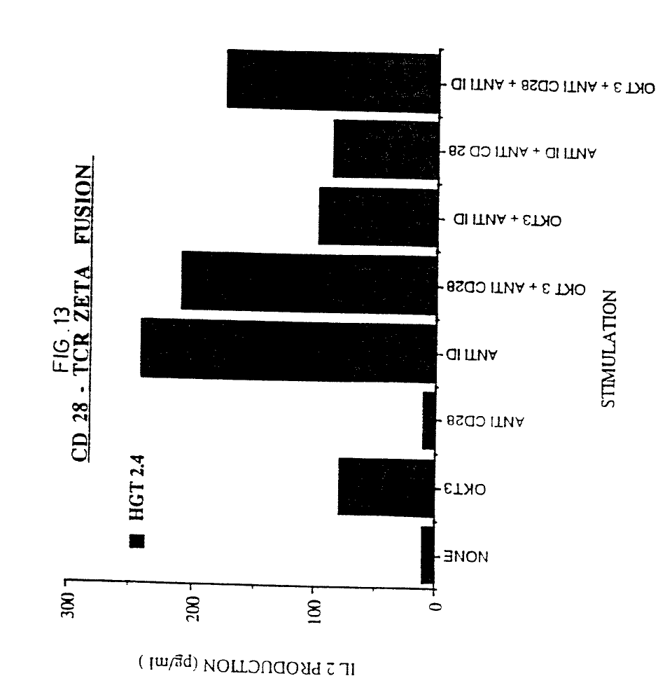

FIG. 13 shows: histogram representation of IL-2 production by HGT2.4 incubated with various combinations of antibodies.

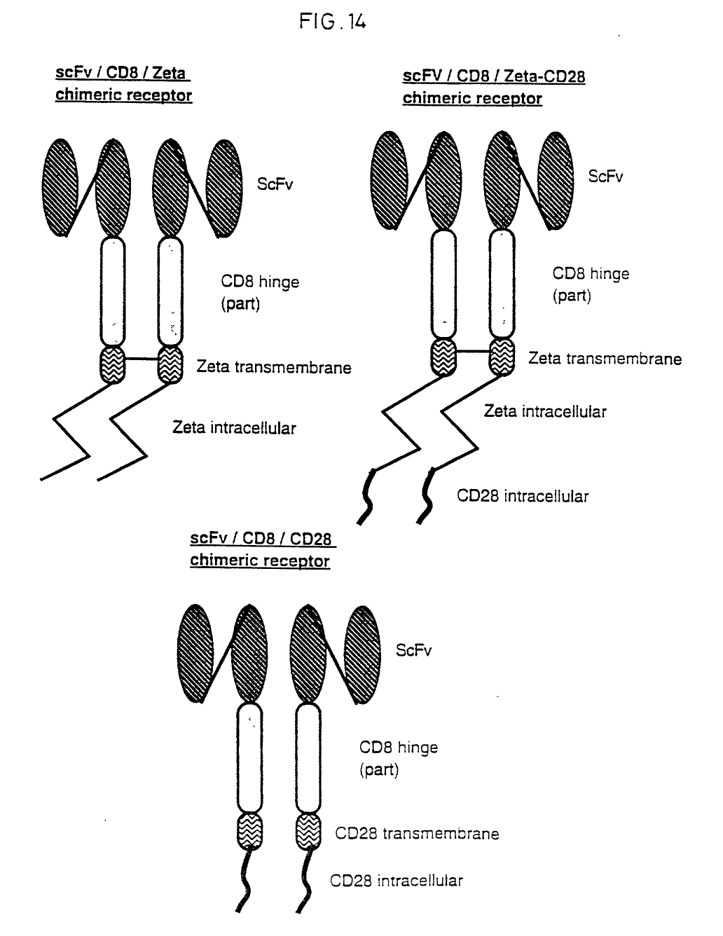

FIG. 14 shows: schematic representation of recombinant chimeric receptor constructs.

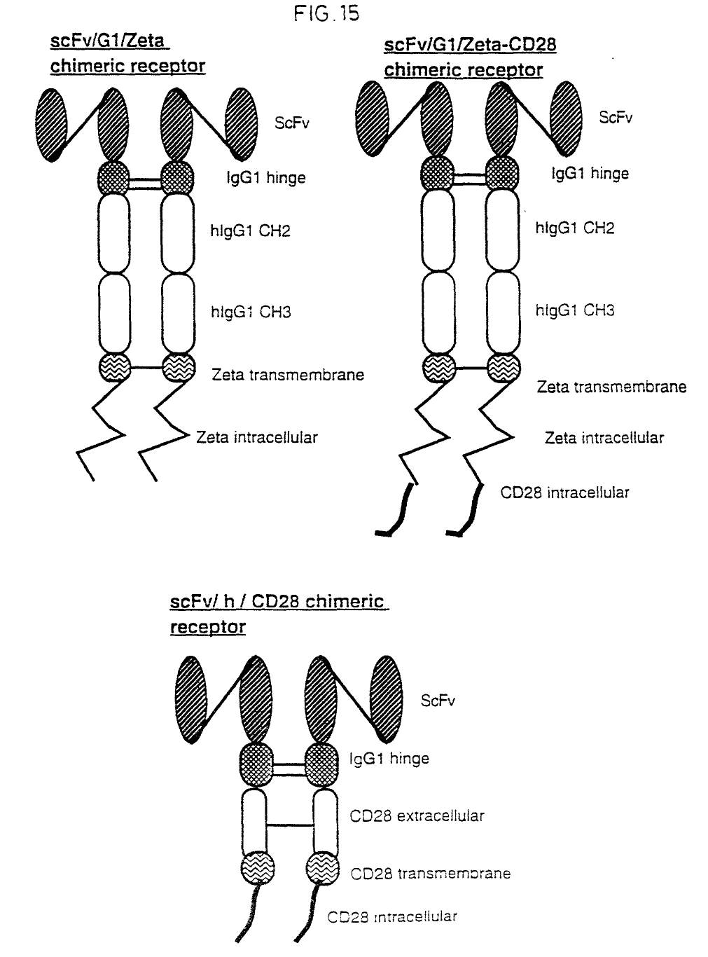

FIG. 15 shows: schematic representation of recombinant chimeric receptor constructs

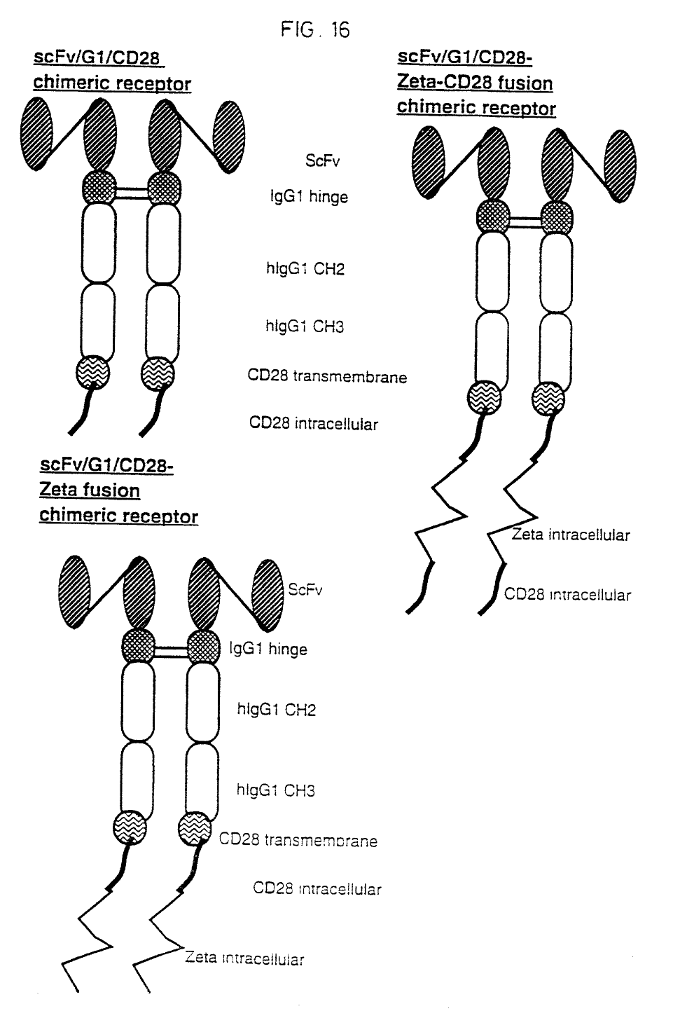

FIG. 16 shows: schematic representation of recombinant chimeric receptor constructs.

FIG. 17 shows: schematic representation of recombinant chimeric receptor constructs

FIG. 18 shows: histogram representation of levels of expression of CD28 chimeras in Jurkat cells

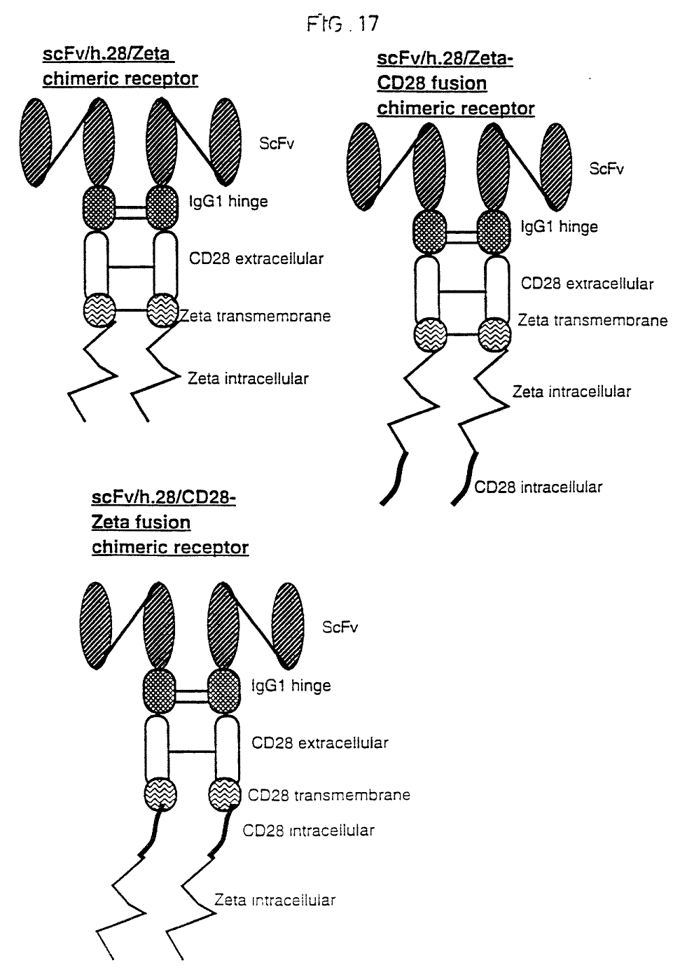

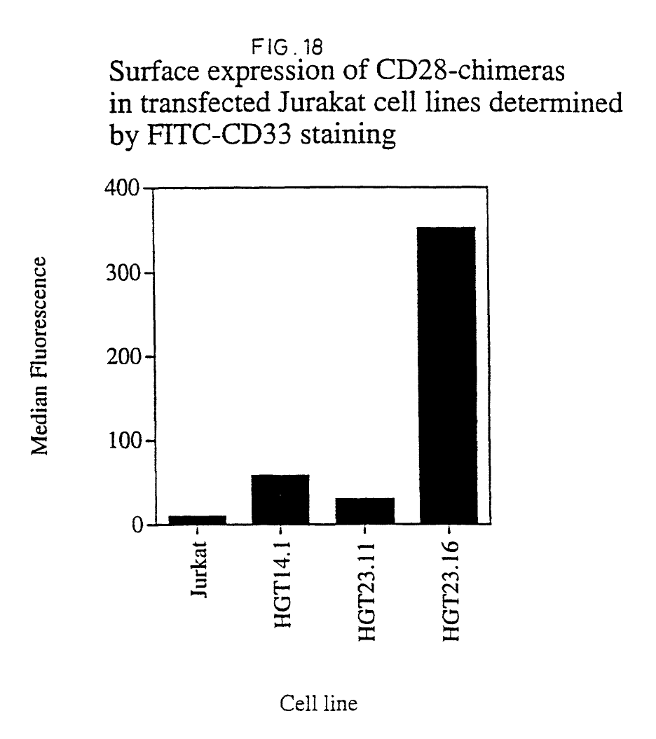

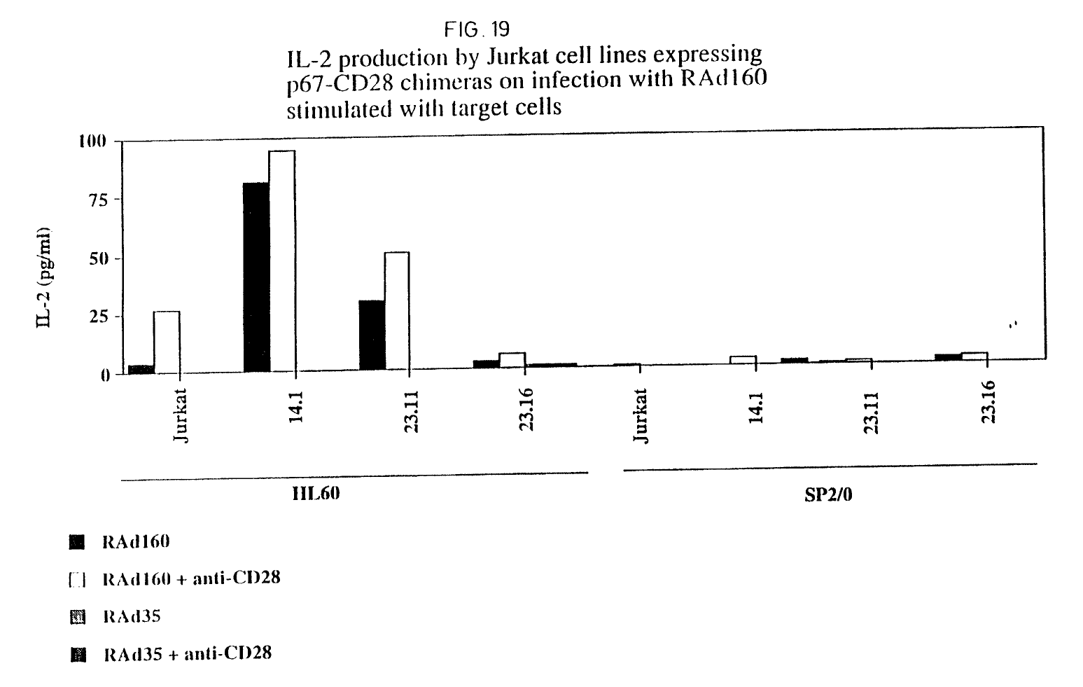

FIG. 19 shows: histogram representation of IL-2 production by Jurkat cells expressing two different chimeric receptors in response to target cells.

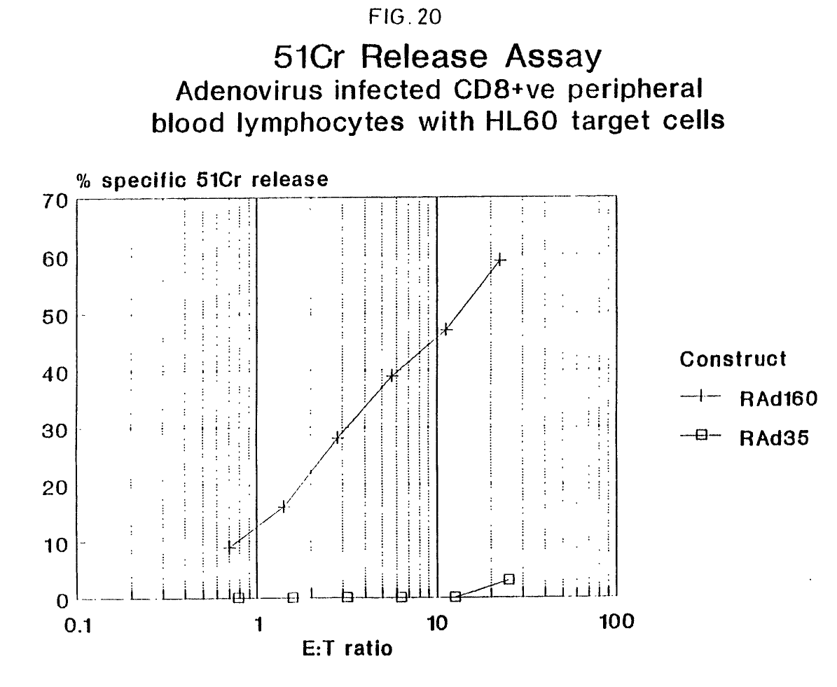

FIG. 20 shows: Graph showing Cytolysis of target cells by CD8+ve human CTL cells infected with recombinant adenoviruses

### EXAMPLE 1

### Construction of Chimeric Receptor Genes

Each component of the chimeric receptor constructs was either PCR cloned or PCR assembled by standard techniques (PCR Protocols, Innis et al, 1990, Academic Press inc.) and sub-cloned in a cassette format into pBluescript SK+ (Stratagene), see FIG. 1, 2, 2b and 2c. Oligonucleotides are described in FIG. 3.

1. Single Chain Fv Cassettes

hCTMO1

An scFv from the engineered human CTMO1 antibody was constructed as follows. Leader sequence and hCTMO1 VI was PCR cloned from plasmid pAL 47 (International Patent Specification No. WO 93/06231) with oligos R6490 and R6516 (Oligo sequences are shown in FIG. 3). R6490 introduces 5′ Not I and Hind III sites and R6516 forms part of the (Gly4Ser)₅ linker. hCTMO1 Vh was PCR cloned from plasmid pAL 52 (WO 93/06231) with oligos R6515 (forms part of linker) and R6514 (introduces 3′ Spe I site. Leader/Vl and Vh fragments were then PCR spliced together and the PCR product was restricted with Not I and Spe I and sub-cloned into pBluescript SK+.

hP67.6

An scFv from another engineered human antibody, hP67.6, engineered according to WO91/09967, was similarly prepared and subcloned into pBluescript SK+.

2. CD8 Hinge Spacer Cassette

The CD8 hinge spacer for hCTMO1 TCR Zeta chimeric receptor and hCTMO1 TCR Zeta-CD28 fusion chimeric receptor (which includes a small part of 5′ Zeta) was PCR assembled using overlapping oligos: R6494,R6495,R6496 and R6497. The CD8 hinge spacer for hCTMO1 CD28 chimeric receptor was PCR assembled using overlapping oligos: R6494,R6495,R6496 and R6506. Both PCR products were restricted with Spe I and BamH I and sub-cloned into pBluescript SK+.

3. Human TCR Zeta Cassette

Human Zeta transmembrane and intracellular components were PCR cloned from human leukocyte cDNA (Clonetech) with oligos R6488 (introducing a 5′ BamH I site) and R6489 (introducing a 3′ EcoR I site). PCR product was restricted with BamH I and EcoR I and sub-cloned into pBluescript SK+.

4. Human CD28 Cassette

Human CD28 transmembrane and intracellular components were PCR cloned from human leukocyte cDNA (Clonetech) with oligos P3240 (introducing a 5′ BamH I site) and P3241 (introducing a 3′ EcoR I site). PCR product was restricted with BamH I and EcoR I and sub-cloned into pBluescript SK+.

5. Hinge-CD28 Cassette

Human CD28 extracellular, transmembrane and intracellular components were PCR cloned from human leukocyte cDNA (Clonetech) with oligos S0146 (introducing a 5′ Spe I site) and P3241 (introducing a 3′ EcoR I site). S0146 also constitutes residues 234 to 243 of human IgG1 hinge. The product of the PCR reaction was digested with restriction enzyme Spe1 and EcoR1 and sub-cloned into pBluescriptSK+.

6. Zeta-CD28 Fusion Cassette

The 3′ end of Zeta, starting at a naturally occuring Sty I site and the intracellular component of human CD28 were PCR assembled such that the Zeta stop codon was removed and an inframe fusion protein would be translated. PCR assembly carried out with overlapping oligos: P3301, P3302, P3303, P3304, P3305 and P3306. PCR product was restricted with Sty I and EcoR I and sub-cloned into pBluescriptSK+ containing the hCTMO1 TCR Zeta chimeric receptor construct, replacing the 3′ end of Zeta.

7. Human IgG1 Spacer Cassette

Human IgG1 hinge, CH2 and CH3 were PCR cloned from IgG1 cDNA clone (A. Popplewell) with oligos S0060 (introducing a 5′ Spe I site) and S0061 (introducing residues L, D, P, and K constituting a 3′ BamH I site). PCR product was restricted with Spe I and BamH I and sub-cloned into pBluescriptSK+.

8. h.28 Spacer Cassette

Human IgG1 hinge and part of human CD28 extracellular component were PCR cloned from a scFv/h/CD28 plasmid with oligos T4057 and T4058. T4057 introduces a 5′ Spe I site and T4058 introduces residues L, D, P, and K constituting a 3′ BamH I site. PCR product was restricted with Spe I and BamH I and sub-cloned into pBluescriptSK+.

9. CD28-Zeta Fusion Cassette

Human CD28 transmembrane and intracellular componenets were PCR cloned from a scFv/h/CD28 plasmid with oligos T7145 and T4060. T7145 introduces residues L, D, P, and K constituting a 3′ BamH I site. T4060 comprises a 3′ overhang compatable with the 5′ end of human Zeta intracellular component.

Human Zeta intracellular component was PCR cloned from a scFv/G1/Zeta plasmid with oligos T4387 and S4700. T4387 comprises a 5′ overhang compatable with the 3′ end of hunan CD28 intracellular component. S4700 introduces a 3′ EcoR I site.

CD28 transmembrane and intracellular components were then PCR spliced to Zeta intracellular component with oligos T7145 and S4700. PCR product was restricted with BamH I and EcoR I and sub-cloned into pBluescriptSK+.

10. CD28-Zeta-CD28 Fusion Cassette

A Pst I restriction site in human Zeta was used to subclone the 3′ end of Zeta intracellular component and the CD28 intracellular component on a Pst I to EcoR I fragment ifrom the Zeta-CD28 fusion cassette into the CD28-Zeta fusion cassette, replacing the 3′ end of Zeta. This generates a CD28-Zeta-CD28 fusion cassette with a 5′ BamH I site and 3′ EcoP I site.

All of the above cassettes were completely sequenced (Applied Biosystems, Taq DyeDeoxy Terminator Cycle Sequencing, Part Number 901497) in pBluescriptSK+ prior to cloning into the expression vectors.

These cassettes were assemled to construct chimeric receptors with the specificity of the engineered human antibodies hCTMO1, directed against human polymorphic epithelial mucin (PEM) or hP67.6, directed against human CD33, by assembling the appropriate cassettes using standard molecular biology techniques. The following chimeric receptors were constructed; see Table 2 and FIGS. 14-17 in which potential di-sulphide bonds are indicated by a horizontal line between the two sub-units (not all di-sulphide bonds may form in 100% of the molecules).

1) scFv/CD8/Zeta Chimeric Receptor (FIG. 14)

The scFv/CD8/Zeta chimeric receptor consists of a single chain Fv (scFv) linked to an extracellular spacer in the form of part of human CD8 hinge, linked to the extracellular, transmembrane and intracellular components of the human T-cell receptor Zeta chain (TCR).

The scFv consists of the leader sequence and variable component of the light chain of the engineered human antibody linked via a (Gly₄Ser)₅ linker to the variable component of the heavy chain of the engineered human antibody. The extracellular spacer consists of residues 98 to 142 of the hinge region of human CD8 (Zamoyska et al: Cell 43, 153-163, 1985). This is linked to residues 6 to 142 of human TCR Zeta comprising extracellular (part), transmembrane and intracellular regions (Weissman et al: PNAS 85, 9709-9713, 1988. Moingeon et al: Eur. J. Immunol. 20, 1741 -1745, 1990).

2) scFv/CD8/CD28 Chimeric Receptor (FIG. 14)

The CD8 hinge/CD28 chimeric receptor consists of a scFv linked to an extracellular spacer in the form of part of human CD8 hinge, linked to the transmembrane and intracellular component of human CD28.

The scFv consists of the leader sequence and variable component of the light chain of the engineered human antibody linked via a (Gly₄Ser)₅ linker to the variable component of the heavy chain of the engineered human antibody. The extracellular spacer consists of residues 98 to 142 of the hinge region of human CD8 (Zamoyska et al: Cell 43 153-163, 1985). This is linked to residues 132 to 202 of human CD28 comprising the transmembrane and intracellular components (Aruffo & Seed: PNAS 84, 8573-8577).

3) scFv/CD8/Zeta-CD28 Fusion Chimeric Receptor (FIG. 14)

The scFv/CD8/Zeta-CD28 Fusion chimeric receptor consists of a single chain Fv linked to an extracellular spacer in the form of part of human CD8 hinge, linked to the extracellular, transmembrane and intracellular components of human TCR Zeta fused to the intracellular component of human CD28.

The single chain Fv consists of the leader sequence and variable component of the light chain of the engineered human antibody linked via a (Gly₄Ser)₅ linker to the variable component of the heavy chain of the engineered human antibody. The extra cellular spacer consists of residues 98 to 142 of the hinge region of human CD8 (Zamoyska et al: Cell, 43, 153-163, 1985). This is linked to residues 6 to 142 of human TCR Zeta comprising extracellular (part), transmembrane and intracellular components (Weissman et al: PNAS 85 9709-9713, 1988 Moingeon et al: Eur. J. Immunol. 20, 1741-1745, 1990). This is linked to residues 162 to 202 comprising the intracellular component of human CD28.

4) scFv/G1/Zeta Chimeric Receptor (FIG. 15)

The scFv/G1/Zeta chimeric receptor consists of a single chain Fv linked to an extracellular spacer comprising human IgG 1 hinge, CH2 and CH3, linked to the transmembrane and intracellular regions of human TCR Zeta.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge, 244 to 360 of CH2 and 361 to 478 of CH3 (Kabat et al Sequences of proteins of immunological interest, 1987). This is linked to residues 6 to 142 of human TCR Zeta comprising extracellular (part), transmembrane and intracellular regions (Weissman et al: PNAS 85, 9709-9713, 1988. Moingeon et al: Eur. J. Immunol. 20, 1741-1745, 1990).

5) scFv/G1/Zeta-CD28 Fusion Chimeric Receptor (FIG. 15)

The scFv/G1/Zeta chimeric receptor consists of a single chain Fv linked to an extracellular spacer comprising human IgG 1 hinge, CH2 and CH3, linked to the transmembrane and intracellular regions of human Zeta fused to the intracellular region of human CD28.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge, 244 to 360 of CH2 and 361 to 478 of CH3 (Kabat et al Sequences of proteins of immunological interest, 1987). This is linked to residues 6 to 142 of human TCR Zeta comprising extracellular (part), transmembrane and intracellular regions (Weissman et al: PNAS 85, 9709-9713, 1988. Moingeon et al: Eur. J. Immunol. 20, 1741-1745, 1990). This is linked to residues 162 to 202 comprising the intracellular component of human CD28 (Aruffo & Seed: PNAS 84, 8573-8577).

6) scFv/h/CD28 Chimeric Receptor (FIG. 15)

The scFv/h/CD28 chimeric receptor consists of a single chain Fv linked to an extracellular spacer consisting of human IgG1 hinge and part of the extracellular region of human CD28, linked to the transmembrane and intracellular regions of human CD28.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge and residues 118 to 134 of human CD28. This is linked to residues 135 to 202 of human CD28 comprising the transmembrane and intracellular regions (Aruffo & Seed: PNAS 84, 8573-8577).

7) scFv/G1/CD28 Chimeric Receptor (FIG. 16)

The scFv/G1/Zeta chimeric receptor consists of a single chain Fv linked to an extra cellular spacer comprising human IgG 1 hinge, CH2 and CH3, linked to the transmembrane and intracellular regions of human CD28.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge, 244 to 360 of CH2 and 361 to 478 of CH3 (Kabat et al Sequences of proteins of immunological interest, 1987). This is linked via residues L, D, P and K to residues 135 to 202 comprising the transmembrane and intracellular components of human CD28 (Aruffo & Seed: PNAS 84, 8573-8577).

8) scFv/G1/CD28 -Zeta Fusion Chimeric Receptor (FIG. 16)

The scFv/G1/Zeta chimeric receptor consists of a single chain Fv linked to an extracellular spacer comprising human IgG 1 hinge, CH2 and CH3, linked to the transmembrane and intracellular regions of human CD28 fused to the intracellular region of human Zeta.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge, 244 to 360 of CH2 and 361 to 478 of CH3 (Kabat et al Sequences of proteins of immunological interest, 1987). This is linked via residues L, D, P and K to residues 135 to 202 comprising the transmembrane and intracellular components of human CD28. This is linked to residues 31 to 142 of human TCR Zeta, the intracellular region (Weissman et al: PNAS 85, 9709-9713. 1988. Moingeon at al: Eur. J. Immunol. 20, 1741-1745, 1990).

9) scFv/G1/CD28-Zeta -CD28 Fusion Chimeric Receptor (FIG. 16)

The scFv/G1/Zeta chimeric receptor consists of a single chain Fv linked to an extracellular spacer comprising human IgG 1 hinge, CH2 and CH3, linked to the transmembrane and intracellular regions of human CD28 fused to the intracellular region of human Zeta fused to the intracellular region of CD28.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge, 244 to 360 of CH2 and 361 to 478 of CH3 (Kabat et al Sequences of proteins of immunological interest, 1987). This is linked via residues L, D, P and K to residues 135 to 202 comprising the transmembrane and intracellular components of human CD28. This is linked to residues 31 to 142 of human TCR Zeta, the intracellular region (Weissman et al: PNAS 85, 9709-9713, 1988. Moingeon et al: Eur. J. Immunol. 20, 1741-1745, 1990). This is linked to residues 162 to 202 comprising the intracellular component of human CD28.

10) scFv/h.28/Zeta Chimeric Receptor (FIG. 17)

The scFv/h/CD28 chimeric receptor consists of a single chain Fv linked to an extracellular spacer consisting of human IgG1 hinge, part of the extracellular region of human CD28 and 4 amino acid residues, linked to the transmembrane and intracellular regions of human TCR Zeta.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge and residues 118 to 134 of human CD28. This is linked via residues L, D, P and K to residues 10 to 142 of human TCR Zeta comprising the transmembrane and the intracellular region (Weissman et al: PNAS 85, 9709-9713, 1988. Moingeon et al: Eur. J. Immunol. 20, 1741-1745, 1990).

11) scFv/h.28/Zeta-CD28 Fusion Chimeric Receptor (FIG. 17)

The scFv/h/CD28 chimeric receptor consists of a single chain Fv linked to an extracellular spacer consisting of human IgG1 hinge, part of the extracellular region of human CD28 and 4 amino acid residues, linked to the transmembrane and intracellular regions of human Zeta fused to the intracellular region of human CD28.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge and residues 118 to 134 of human CD28. This is linked via residues L, D, P and K to residues 10 to 142 of human TCR Zeta comprising transmembrane and intracellular regions (Weissman et al: PNAS 85, 9709-9713, 1988. Moingeon et al: Eur. J. Immunol. 20, 1741-1745, 1990). This is linked to residues 162 to 202 comprising the intracellular component of human CD28.

12) scFv/h.28/CD28-Zeta fusion Chimeric Receptor (FIG. 17)

The scFv/h/CD28 chimeric receptor consists of a single chain Fv linked to an extracellular spacer consisting of human IgG1 hinge, part of the extracellular region of human CD28 and 4 amino acid residues, linked to the transmembrane and intracellular regions of human CD28 fused to the intracellular region of human Zeta.

The single chain Fv consists of the leader sequence and variable component of the light chain of the engineered human antibody linked via a (Gly₄Ser)₅ linker to the variable component of the heavy chain of the engineered human antibody. The extracellular spacer consists of residues 234 to 243 of human IgG1 hinge and residues 118 to 134 of human CD28. This is linked via residues L, D, P and K to residues 135 to 202 comprising the transmembrane and intracellular components of human CD28. This is linked to residues 31 to 142 of human TCR Zeta, the intracellular region (Weissman et al: PNAS 85, 9709-9713, 1988. Moingeon et al: Eur. J. Immunol. 20, 1741-1745, 1990).

Table 1 shows a number of preferred recombinant chimeric receptors which may be made in an analogous way by following the above teaching and methods.

Table 2 gives details of the chimeric receptor constructs and cell line nomenclature used.

### EXAMPLE 2

### Analysis of hCTMO1-Chimeric Receptor Constructs Expressed in Jurkat Cells

Chimeric receptor constructs were sub-cloned from pBluescriptSK+ into the expression vectors pEE6hCMV.ne and pEE6hCMV.gpt (Bebbington (1991), Methods 2, 136-145) on a Hind III to EcoR I restriction fragment. The hCTMO1/CD8/Zeta chimeric receptor construct was cloned into pEE6hCMVne and the hCTMO1/CD8/CD28 and hCTMO1 Zeta-CD28 fusion chimeric receptor constructs were cloned into pEE6hCMVgpt.

Plasmids were linearised and transfected into Jurkat E6.1 cells (ECACC) by electroporation using a Bio-Rad Gene Pulser using the method of Rigley et al (J. Immunol. (1995) 154, 1136-1145). Chimeric-receptor expressing colonies were selected in media either containg the drug G418 (2 mg/ml) for Neo vectors or Mycophenolic acid for Gpt vectors as described (Rigley et al ibid.). After approximately four weeks colonies were visible. Colonies were screened by analysis of surface expression of single chain Fv.

Antibodies

Anti-idiotype antibodies are purified antisera from rabbits immunised with hCTMO1. Anti-Id antibodies were purified initially on Protein A-Sepharose absorbed out against human IgG-Sepharose and finally affinity purified on hCTMO1. OKT3 recognises an extracellular compononent of human CD3 ε (ATCC). Anti-CD28 used in these experiments was a rat IgG2b monoclonal antibody (clone YTH 913.12) directed against the extracellular component of human CD28 (Cymbus Bioscience). FITC labelled donkey anti-rabbit Ig recognises rabbit heavy and light chains (Jackson Research Laboratories).

Analysis of Surface Expression of scFv

Approximately 5×10⁵ cells were stained with saturating concentrations of anti-idiotype (10 μg/ml), then incubated with fluorescein-conjugated donkey anti-rabbit antibody. Fluorescence was analysed by a FACScan cytometer (Beckton Dickinson).

Anti-Id Stimulation

1×10⁶ Jurkat transfectants were incubated in a 96 well plate (Nunc) previously coated with/without a saturating concentration of anti-idiotype antibody at 37° C./5% CO₂ in non-selective media. Additional stimuli of anti-CD28 and OKT3 were added in solution to a final concentration of 5 μg/mL. After 18 to 20 hours cells were centrifuged and supernatant assayed for human IL-2 (Quantikine kit, R & D Systems).

Antigen Expressing Cell Stimulation

1×10⁶ Jurkat transfectants were incubated with 1×10⁵ MCF-7 cells (P.E.M. antigen expressing) in a 96 well plate (Falcon) overnight at 37° C./5% CO₂.

Additional stimulus of anti-CD28 was added in solution to a final concentration of 5 μg/mL. After 18 to 20 hours cells were centrifuged and supernatant assayed for human IL-2 (Quantikine kit, R & D Systems).

RESULTS

Cross-linking the T-cell receptor with anti-CD3 antibodies can be used to stimulate human T-cell lines such as Jurkat E6.1 to produce cytokines including IL-2. The expression of IL-2 can be further enhanced by co-stimulation by means of antibodies to the CD28 cell surface molecule in this cell line. This therefore provides a convenient model system to evaluate chimeric receptors for the ability to deliver signals which are co-stimulatory for T-cell activation.

1. Enhancement of IL2 Production by a Jurkat E6.1 Cell Line Transfected with an hCTMO1 scFv-CD8-TCR ζ Chimeric Receptor (Plasmid pTB3 in Response to Antigen or Anti-idiotype Antibody by Co-stimulation with an Anti-CD28 Antibody.

The cell lines TB 3.2, 3.13 and 3.24 were stable cell lines derived from Jurkat E6.1 transfected with CTMO1hscFv/CD8/Zeta. FIG. 10 shows IL2 production by these cell lines when stimulated with an anti-CTMO1 idiotypic antibody alone or in combination with an anti-CD28 antibody. In each case the co-stimulation with anti CD-28 results in a greater than 2-fold stimulation of IL2 production compared to stimulation with anti-CTMO1 idiotype antibody alone. Incubation of these cell lines with anti-CD28 alone did not result in stimulation of IL2.

FIG. 11 shows the production of IL2 by one of the above cell lines (TB 3.13) when stimulated with antigen expressing tumour cells. As in FIG. 10 this is shown with and without co-stimulation using anti-CD28 antibody and indicates that co-stimulation can enhance IL-2 production when stimulation of the chimeric receptor is mediated by antigen.

2. Construction and Testing of a Chimeric Receptor Designed to Generate a Response Analogous to CD28 Stimulation on Interaction with the Extracellular scfv Component.

Having established that co-stimulation via the CD28 molecule could result in enhancement of the response of a T cell transfectant to a tumour associated antigen a chimeric receptor incorporating the CD28 transmembrane and cytoplasmic components was constructed. This hCTM01/CD8/CD28 chimeric receptor (pHMF332) (HGT1) was transfected into Jurkat E6.1 cells to generate stable cell lines. Two of these lines HGT 1.2 and 1.4 were incubated in the presence of various combinations of stimulating antibodies as shown in FIG. 12 (see materials and methods for experimental procedure), and anti-idiotypic antibody was used to stimulate the chimeric receptor.

Incubation of the cell lines shown with an anti-CD3 antibody resulted in a low level of IL2 production. This stimulation could be enhanced by co-stimulating with an anti-CD28 antibody (column 5 FIGS. 12a and 12b).

Incubation with the anti-CD28 alone as expected did not result in IL2 production.

Similarly incubation with the anti-idiotypic antibody alone (stimulating the chimeric CD28 receptor) resulted in no IL2 production. However, by analogy with the combined anti-CD3 and anti-CD28 stimulation, incubation with anti-CD3 and anti-idiotype resulted in IL2 production enhanced over CD3 stimulation alone. This demonstrates that a chimeric receptor could be constructed that responds via stimulation of extracellular scFv to generate an intracellular signal capable of costimulating CD3 mediated activation.

3. Provision of Both Primary and Accessory Stimulation in the Same Effector Cell.

In order to provide both primary (for example TCR ζ mediated) and co-stimulatory (for example CD28 mediated) activation of the effector cell via interaction of a chimeric receptor with a defined ligand or antigen a fusion receptor incorporating two different signalling components was constructed. This chimeric receptor hCTMO1/CD8/TCRZeta-CD28 (pHMF334) was transfected into Jurkat E6.1 cells and stable lines selected. One of these lines (HGT 2.4) was incubated with various combinations of antibodies and IL2 production measured (see FIG. 13).

The anti-CD3 and anti-CD28 antibodies individually and in combination resulted in a similar relative stimulation of IL2 production to that seen with the other transfected cell lines. However, with the construct HGT2 the anti-idiotype antibody alone resulted in a level of IL2 production greater than achieved with the combined anti-CD3 and anti-CD28 antibodies. Furthermore, the stimulation achieved with the single anti-idiotypic interaction could not be enhanced by further co-stimulation with anti-CD3. anti-CD28 or combinations of these.

### EXAMPLE 3

### Analysis of Single Gene hP67.6-Chimeric Receptor Constructs Expressed in Jurkat Cells

In order to confirm the results obtained with the hCTMO1 fusion receptor for a different antibody scFv, and to evaluate additional fusion receptors, a number of different chimeras based on the hP67.6 scFv were introduced into Jurkat cells.

Chimeric receptor constructs hP67.6/G1/Zeta (HGT16), hP67.6/G1/Zeta-CD28 (HGT17), hP67.6/G1/CD28-Zeta (HGT21), hP67.6/G1/CD28-Zeta-CD28 HGT26), hP67.6/h.28/Zeta-CD28 (HGT20) and hP67.6/h.28/CD28-Zeta (HGT22) chimeric receptor constructs were sub-cloned from pBluescriptSK+ into the expression vector pEE6hCMV.ne as described in Example 2. Expression plasmids were transfected into Jurkat E6.1 and permanent cell lines expressing chimeric receptors on their cell surfaces were identified as described above (Example 2) but using a purified rabbit anti-p67.6 idiotye antiserum prepared as described for hCTMO1 anti-idiotype. Alternatively, cells were stained with purified recombinant CD33 extracellular domain conjugated to FITC (10 μg/ml) and analysed directly on the cytometer.

Western blot analysis was carried out on representative clones for each construct to confirm that chimeric recptors of the expected size were expressed. Approximately 10⁷ cells were lysed in lysis buffer (1% NP40, 150 mM NaCl, 10 mM NaF, 0.4 mM EDTA, 1 mM Na vanadate, 1 mg/ml Pefabloc, 10 μg/ml Pepstatin, 10 μg/ml Leupeptin, 20 μg/ml Aprotinin) and samples subjected to SDS-PAGE with or without reduction of cystine residues with β-mercaptoethanol. Western blots were probed with rabbit ant-P67.6 idiotype followed by horseradish-peroxidase (HRP) conjugated donkey anti-rabbit Ig or HRP-conjugated rabbit anti-human Fc antisera according to standard techniques.

A comparison of the apparent molecular weights of the chimeric receptors in reduced and non-reduced samples indicated that the zeta-chain chimera in cell line HGT16.1 and the fusion receptor in HGT17.39 were present as di-sulphide linked homodimers. The CD28 chimera in HGT14.1 is present as approximately 50% disulphide-linked homodimers and approximately 50% of the molecules are not disulphide linked. At least 50% of molecules are disulphide-linked in the case of the fusion receptors in HGT20, HGT21 and HGT22 cell lines.

A panel of independent transfectant clones for each construct were analysed for IL-2 production in response to cells which express CD33 (HL60 cells) or are CD33 negative (eg Jurkat E6.1). It is important to analyse a number of clones expressing each construct since individual clones vary substantially in the level of expression of chimeric recptor. Moreover, even clones expressing similar levels of receptor show different capacities to produce IL-2. Each transfectant was mixed with an equal number of target cells (eg 10⁵ cells of each cell type per well of a 96-well plate) and co-cultured for approximately 20 h. The concentration of IL-2 in the supernatant was then determined using a Quantikine human IL-2 ELISA (R&D Systems).

Cell lines containing construct HGT 16 produce levels of IL-2 in response to HL60 cells of up to approximately 200 pg/ml and do not produce detectable IL-2 when stimulated with CD33-negative cells. Cell lines expressing fusion receptors HGT17, 20, 21, 22 and 26 also produce IL-2, specifically in response to CD33 positive target cells, indicating that the zeta-chain signalling capacity is intact in the fusion proteins. In fact cells expressing the fusion receptors at comparable levels on the cell surface produce on average more IL-2 in response to HL60 cells than HGT16 cell lines (from 50% more to 7-fold more), consistent with their capacity to provide both primary and co-stimulatory signals.

The function of the CD28 signalling domain can be confirmed by assaying for recruitment of downstream signalling components to the CD28 intracellular domain in response to receptor ligand binding. The association of the regulatory (p85) sub-unit of P13-kinase with phosphorylated ITAM motifs of the sequence YMXM (single-letter amino acid code) in the CD28 intracellular domain in response to CD28 stimulation is well documented (eg Stein et al., 1994 Mol. Cell. Biol. 14: 3392-3402). CD28 also associates specifically with the tyrosine kinase ITK on activation (August et al. 1994 Proc. Natl. Acad. Sci. USA 91: 9347-9351).

Association of p85 with the receptor chimeras is analysed by immunoprecipitation of the receptor and detection of bound p85 protein by Western blotting as follows. Approximately 5×10⁷ cells are washed once with PBS and activated in 0.5 ml PBS containing 10 μg/ml rabbit anti-P67.6 idiotype antibody at 37° C. for various times from 0-10 mins. Cells are then washed twice with ice-cold PBS and lysed in 1 ml lysis buffer as described above. Lysates are centrifuged at 15000 rpm in an Eppendorf micro-centrifuge for 10 min. and the supernatants immunoprecipitated with 100 μl protein A-sepharose beads (Pharmacia) at room tempeature for 30 min. (This immunoprecipitation procedure also serves to immunoprecipitate chimeric receptors containing antibody constant regions from cells which have not been stimulated with anti-idiotype antibody to act as a negative control). The beads are then washed 3 times with fresh lysis buffer, resuspended in 50 μl SDS loading buffer and subjected to SDS-PAGE and Western blotting. Blots are probed with mouse anti-p85 monoclonal antibody and HRP-conjugated rabbit anti-mouse Ig according to standard techniques.

This showed that p85 can associate with fusion receptors but not with the zeta chain chimera in cell line HGT16.1 thus confirming that p85 associates specifically with CD28 and not zeta and that CD28 signalling is retained in fusion chimeras.

Association of ITK with CD28 intracellular components is detected using published methods (August et al. 1994 Proc. Natl. Acad. Sci. USA 91: 9347-9351).

### EXAMPLE 4

### Expression of Two hP67.6-Chimeric Receptors in the Same Cell

In order to express both a zeta chimeric receptor and a CD28 co-stimulatory receptor chimera in the same cell, stably transfected Jurkat cell lines expressing CD28 receptor chimeras were infected with recombinant adenovirus encoding the hP67.6/G1/Zeta chimeric receptor.

The hP67.6/h.28/CD28 construct was sub-cloned into pEE6hCMV.gpt and transsfected into Jurkat E6.1 cells as described in Example 2. Cell line HGT14.1 is a Jurkat trensfectant expressing this construct. The hP67.6/G1/CD28 construct was cloned into pEE6hCMV.ne and Jurkat clones HGT23.11 and HGT23.16 expressing this construct were isolated as in Example 2. The levels of expression of the CD28 chimeras on the surface of the transfected cells, determined by FAC-analysis with FITC-CD33 as described in Example 3, is shown in FIG. 18.

In order to transiently express a uniform amount of the zeta-chain chimera hP67.6/G1/zeta in each of these CD28-chimera cell lines, a recombinant adenovirus vector expressing the zeta chimera was constructed as follows. The hP67.6/G1/zeta coding sequence from pHMF342 (Example 1 and Table 2) was excised as a Not1-Kpn1 fragment and inserted into the adenovirus-5 transfer vector pAL119 (provided by G. Wilkinson. Department of Medicine, University of Wales, Cardiff; unpublished) between the Not1 and BamH1 sites, after insertion of a Kpn1-BamH1 adaptor oligonucleotide, to form pAL119-342. In this plasmid, the chimeric receptor coding sequences are expressed under the control of the hCMV-MIE promoter-regulatory region and polyadenylation signal (Wilkinson and Akrigg 1992 Nucl. Acids Res.20: 2233-2239).

Suitable alternative adenovirus transfer vectors containing the hCMV-MIE promoter include pCA3 and pCA4 (Hitt et al. 1995 in Methods in Molecular Genetics, K. W. Adolph (ed) Academic Press, Orlando.) Alternative adenovirus transfer vectors can be used such as pAC (Gerard and Meidell 1995 In DNA Cloning: a practical approach (2nd edition) Volume 4 ed Glover and Hames, IRL Press) which does not contain a promoter. In this case, one of many other heterologous promoters, such as the RSV-LTR promoter or T-cell specific promoters, may be introduced upstream of the chimeric receptor coding sequence prior to insertion into the transfer vector. Additional RNA processing signals are also desirable, such as a polyadenylation signal (eg from SV40 Virus) and an intron (e.g. from the hCMV-MIE gene) (Bebbington (1991), Methods 2, 136-145).

Approximately 5 μg pAI119-342 was co-transfected with 5 μg pJM17 (Microbix Biosystems Inc., McGrory et al. 1988 Virology 163: 614-617) into the human embryonic kidney cell line, 293 (ATCC CRL 1573) by calcium phosphate-mediated transfection, according to standard procedures for construction of adenovirus recombinants (Lowenstein et al 1996 in Protocols for gene transfer in Neuroscience, P. R. Lowenstein and L. W. Enquist (eds) Wiley and Sons). This generated recombinant virus RAd160 containing the chimeric receptor cDNA under the control of hCMV-MIE gene regulatory regions. Large scale preparations of RAd160 were prepared (Lowenstein et al ibid.) with titres of greater than 10¹⁰ pfu/ml and stored at −70° C. in small aliquots.

Recombinant adenoviruses containing coding sequences for CD28 chimeric receptors are prepared in the same way after insertion of the desired coding sequence into pAL119 or another adenovirus transfer vector.

RAd160 was added to Jurkat E6.1 cells or transfectants expressing CD28 receptor-chimeras at a multiplicity of infection (MOI) of up to 400 pfu/cell with 2 μg/ml DEAE-Dextran and incubated for 24 h at a cell concentration of 10⁶ cells/mi in the presence of virus. Samples of cells were infected with a recombinant adenovirus expressing an irrelevant β-galactosidase protein RAd35 (Wilkinson and Akrigg 1992 Nucl. Acids Res.20: 2233-2239) in the same way to act as a negative control. Infected cells were then washed once in fresh growth medium, expanded in culture for a further 6 days and assayed for IL-2 production in response to target cells. The results are shown in FIG. 19. Jurkat cells infected with RAd160 produce essentially undetectable levels of IL-2 in response to HL60-cell stimulation (less than 10 pg/ml) unless co-stimulated with 10 μg/ml anti-CD28 antibody 15E8 (Caltag) which leads to low levels of IL-2 production specifically in response to HL60 cells and not in response to a cell ine which does not express human CD33, the murine SP2/0 cell line. In contrast, RAd160-infected HGT14.1 cells, which express a CD28 chimeric receptor, produce significant levels of IL-2 specifically in response to HL60 target cells even in the absence of anti-CD28 antibody. This indicates that the CD28-chimeric receptor hP67.6/h.28/CD28 is able to contribute the requisite co-stimulation to the zeta chimera. Cell lines expressing the alternative CD28 chimeric receptor, hP67.6/G1/CD28, 23.11 and 23.16 show markedly reduced levels of IL-2 production compared with 14.1. Indeed, 23.16, the cell line expressing the highest level of this CD28 chimera produces no detectable IL-2 at all. The CD28 signalling pathway was shown to be intact in this cell line since stimulation through CD3 (using anti-CD3 antibody) in 23.16 yields very high levels of IL-2 (results not shown). Thus the signalling defect in cell lines expressing the hP67/G1/CD28 chimera appears to be due to interference with zeta-chain signalling. The mechanism responsible is likely to be related to the use of the same extracellular domain in the zeta and CD28 chimeric receptors. This will allow heterodimerisation of the two receptors and this appears to interfere with zeta-chain signalling. This hypothesis is supported by the fact that 23.16, expressing high levels of the CD28 chimera, shows greater interference with zeta-chain signalling than 23.11, expressing very low levels of the CD28 chimera (FIG. 18).

This experiment shows that it is possible to use the same scFv region to stimulate two chimeric receptor molecules in the same cell, one to provide a primary stimulus in response to antigen and the other receptor to provide a co-stimulatory signal. This leads to efficient IL-2 production specifically in response to antigen-expressing target cells provided that the two receptors are prevented from heterodimerisation, for instance by using different dimerisation domains on the two receptors. It is envisaged that additional pairs of dimerisation domains will be compatible. For instance the scFv/h.28/zeta chimeric receptor (Example 1; FIG. 17) could provide the primary signal and the scFv/G1/CD28 receptor (Example 1: FIG. 16) would provide the co-stimulatory signal.

### EXAMPLE 5

### Identification of Additional Co-stimulatory Cell-surface Receptors Using Anti-receptor Antibodies

5×10⁵ HGT16.1 cells expressing the hP67.6 scFv/G1/zeta chimeric receptor (Example 3) were incubated for 16 h with an equal number of HL60 cells in the presence of various mouse monoclonal antibodies directed against human T-cell surface markers. The bivalent antibodies were included at 10 μg/ml to test for their ability to co-stimulate the zeta-chain chimera. The antibodies used in this experiment were: anti-CD2 RPA2.10 (Pharmingen), anti-CD3 OKT3 (ATCC), anti-CD4 OKT4 (ATCC), anti-CD5 UCHT2 (Pharmingen), anti-CD28 15EB (Caltag) and a control antibody MOPC21 (ATCC). IL-2 accumulated in the supernatant at the end of the incubation was measured by Quantikine IL-2 ELISA (R&D Systems).

The results indicate that anti-CD2, anti-CD5 and anti-CD28 co-stimulate production of IL-2 in HGT16.1 cells in response to HL60 target cells hence confirming CD2, CD5 and CD28 as co-stimulatory receptors compatible with zeta-chain chimera signalling. From experiments designed in this way, it would be possible to determine the co-stimulatory activity of other cell surface molecules. The intracellular domains can then be included in chimeric receptors as described in Example 1 and evaluated as described in Examples 2, 3 and 4.

### EXAMPLE 6

### Introduction of Chimeric Receptors into Primary Human CTLs

In order to establish an assay for co-stimulation of cytolytic T-cell function, a zeta-chain chimera was introduced into primary human T-cells using recombinant adenovirus vectors. Peripheral blood mononuclear cells (PBMC) were isolated from healthy volunteers using centrifugation over Ficoll-Hypaque (Pharmacia) according to the manufacturer's instructions and cultured in RPMI-1640 medium with 10% FCS in 175-cm² tissue culture flasks. Non-adherent cells were transferred to fresh tissue culture flasks after 24 h and phytohaemagglutinin (PHA) was added to a final concentration of 2 μg/ml and human recombinant IL-2 at 50 ng/ml. After 6 days, CD4-positive cells were removed using anti-CD4 antibody immobilised on magnetic Dynabeads (Becton-Dickinson) to leave a population of cells at least 95% CD8-single positive (CTL cells). The cells were washed by centrifugation and resuspended in fresh medium +10% FCS at 10⁶ cells /ml.

Recombinant adenovirus RAd160 (expressing the hP67.6/G1/zeta chimeric receptor, Example 4) or the control virus RAd35 was added to the cells at a multiplicity of infection (MOI) of up to 400 pfu/cell with 2 μg/ml DEAE-Dextran and incubated for 24 h. Samples of cells were then fixed in 1% glutaraldehyde in PBS and infection rates measured by staining RAd35-infected cells for β-galactosidase activity using 5-Bromo-4-chloro-3-indolyl β-D-galactoside (X-gal; Promega, according to the manufacturer's instructions). By this method, infection frequencies were determined to be at least 80%. Infected cells were expanded in culture for a further 6 days in medium containing 50 ng/ml human IL-2. In some experiments, 2 mM sodium butyrate was added to infected CTL cells to induce expression from the hCMV-MIE promoter.

Cytolytic activity against the CD33-expressing tumour cell line HL60 was detected in recombinant adenovirus-infected CD8-positive cells incubated for 6 days in IL-2 and 2 mM butyrate using standard 6 h ⁵¹Cr release assays. 2×10⁷ HL60 target cells were labelled by incubation with 25 MBq ⁵¹Cr (CJS4 Amersham) for 45 min. at 37° C. in T-cell growth medium. After washing, 1.5×10⁴ labelled HL60 cells were transferred into each well of a 96-well microtitre plate in the presence of RAd-infected CD8-positive effector cells at ratios in the range 100 to 0.1 effector:target cells. Cells were incubated for 6h in T-cell growth medium before centrifuging the plates and removal of the supernatant for counting. Cytolysis was expressed as the amount of ⁵¹ Cr released into the medium compared to that released by detergent treatment of target cells. In the experiment illustrated (FIG. 20) specific lysis was mediated by RAd 160-infected effector cells but not by CD8-positive cells infected with RAd35. The degree of specific lysis is increased with increased E:T ratio.

This assay is useful for determining the effects of co-stimulation on cytolytic function using anti-receptor antibodies, co-stimulatory cytokines or co-stimulatory chimeric receptors. Cells starved of IL-2 for various lengths of time can also be used to increase the sensitivity of assays designed to evaluate co-stimulatory activities. CD28 chimeric receptors can be introduced by co-infection of recombinant adenovirus with RAd160. Alternatively a fusion receptor containing both zeta and CD28 signalling domains can be introduced using a single recombinant adenovirus. Anti-receptor antibodies which may be screened in this assay include anti-CD2 and anti-CD5 (see Example 5).

### EXAMPLE 7

### Analysis of Co-stimulatory Activities in Macrophages and Monocytes

Human monocytes were isolated from peripheral blood as follows. PBMC were isolated as described above and adherent cells obtained by settling on to plastic tissue culture flasks for 24 h before washing extensively with fresh medium.

Primary macrophages were isolated from the peritoneal cavity of Wistar rats 5 days after i.p. injection of 5 ml 3% thioglycollate (Sigma T-9032) in saline according to the method of Argys (Argys 1967, J.Immunol. 99:744-750) or 3 ml mineral oil (heavy white oil; Sigma 400-5). Peritoneal lavage was carried out with 20 ml RPMI 1640 medium+10% FCS and 3.15% sodium citrate. Greater than 60% of the cells in the peritoneal lavage were mononuclear phagocytes as defined by flow cytometry using FITC-conjugated mouse anti-rat macrophage antibody ED2 (Serotec) and morphological characteristics. Adherent cells were enriched by applying cells to plastic flasks or 6-well plates in RPMI 1640 medium+10% FCS and culturing for 2 days. Non-adherent cells were then removed by extensive washing with fresh medium. Alternatively, macrophages were purified by Percoll density centrifugation (Lawson and Stevenson 1983 Br. J. Cancer 48: 227-237.)

Monocytes and macrophages were maintained in culture for 48 h and infected with recombinant adenoviruses at a MOI of up to 200 pfu/cell for 16 h in the presence of 2 μg/ml DEAE-Dextran, after which the virus was removed by washing with fresh medium. Up to 80% of human peripheral-blood monocytes and rat peritoneal macrophages were infectable using this procedure, as determined using X-gal staining of cells infected with RAd35. The use of higher concentrations of virus increased the percentage of cells infected but led to a significant reduction in cell viability.

The recombinant adenovirus RAd160 can be used to provide a human CD33-specific primary stimulus to cells of the rat or mouse monocyte-macrophage lineage. Since human monocytes express the CD33 antigen, for the analysis of chimeric receptor function in human monocytic phagocytes, it may be more appropriate to use an alternative binding specificity such as the hCTMO1scFv-containing chimeric receptor, constructed as in Example 1 and inserted into a recombinant adenovirus vector. Additionally, the zeta chain sequences of the chimeric receptor may be substituted with the transmembrane and intracelluar domain of a FcRIII γ chain (Park et al 1993, J. Clin. Invest. 92: 2073-2079).

Rat peritoneal macrophages infected with RAd160 at an MOI of 100 pfu/cell, expressed high levels of chimeric receptor on their surfaces 48 h post-infection as determined by staining with FITC-CD33 and analysis by a FACScan flow cytometer.

The response of monocytes and macrophages expressing the appropriate chimeric receptor to stimulation with specific antigen or antigen-expressing cells recognised by the scFv is measured in standard ⁵¹Cr release assays (Example 6). Alternatively, phagocytosis and cytostasis assays (Lawson and Stevenson 1983 Br. J. Cancer 48: 227-237) or assays for the release of cytokines are carried out eg human TNF ELISA (R&D Systems) or rat TNF ELISA (Biosource).

Identification of appropriate receptor intracellular domains to provide a co-stimulatory signal can be accomplished by incubation of macrophages expressing the chimeric receptor with a source of the specific antigen and with cross-linking antibodies or natural ligands specific for individual cell surface receptors present on monocytes and macrophages as described in Example 5. Suitable receptors include the IL-2 receptor, the CSF-1 receptor, the IFN-γ receptor, the GM-CSF receptor and TNF receptors.

Natural ligands which can be used for human monocytes macrophages include recombinant human IL-2, human CSF-1 (M-CSF), human IFNγ, human GM-CSF and human TNFα (all from Genzyme). Ligands which can be used for rat or mouse macrophages include recombinant rat or human IL-2, human CSF-1 (M-CSF), mouse IFNγ, mouse GM-CSF and mouse TNFα (Genzyme). Species-specific antibodies which cross-link and stimulate the chosen receptors can be raised using standard techniques or can be identified by screening commercially available antibodies.

Those antibodies or natural ligands which co-stimulate macrophage responses to CD33 identify candidate receptors whose intracellular domains or associated signalling molecules, such as receptor-associated tyrosine kinases, can be used to produce chimeric co-stimulatory receptors or fusion receptors containing both co-stimulatory and primary signalling domains as described in Example 1. The intracellular components which may be used in these chimeric recptors include the following. The intracellular domains of the GM-CSF receptor β chain can be used as part of a di-sulphide linked homodimeric receptor or in combination with an intracelluar component from the α chain (Muto et al. 1996, J. Exp. Med. 183: 1911-1916). The intracelluar domains of the IFNγ-receptor α and β chains can be used (Bach et al., 1996.. Mol. Cell. Biol. 16: 3214-3221.), as can the intracellular domains of the IL-2 receptor, particularly the β and γ chains. One or more intracelluar tyrosine kinase components can be used such as the jak1, jak2 and jak3 kinases or the intracellular domain of the CSF-1 receptor tyrosine kinase (Carlberg and Rohrschneider 1994 Mol. Biol. Cell 5:81-95). If these tyrosine kinases are used, the receptors containing them are preferably constructed so that they are presented on the cell surface as monomers which oligomerise on binding of the scFv component to the target antigen, for instance using a scFv coupled to a CD8 hinge extracellular component, coupled to a CD28 transmembrane component (see Example 1) which is coupled to the tyrosine kinase component.

### EXAMPLE 8

### Analysis of Co-stimulatory Activities in Other Cells of the Immune System

Additional immune cell types such as CD4-positive T-cells, B-cells, NK cells, basophils, neutrophils, haematopietic stem cells are isolated from human peripheral blood, mouse or rat blood or peritoneal cavity or other sources by published procedures (Current Protocols in Immunology ed Coligan et al. John Wiley and Sons). Established cell lines which retain the differentiated functions of various immne cell types can also be used eg the human NK-like cell line YT2C2 (Roger et al 1996 Cellular Immunol. 168: 24-32.) A chimeric receptor capable of delivering a primary stimulus such as the hP67.6/G1/zeta chimera described above is introduced into the isolated immune cell type, eg by infection with recombinant adenovirus RAd160, and cross-linking antibodies or natural ligands of cell surface receptors are used to identify cell-surface molecules capable of providing co-stimulatory signals as described in Example 7.

Chimeric receptors containing appropriate cytoplasmic components to provide suitable co-stimulatory functions are then constructed as described in Example 1. The function of the chimeric receptors in the chosen cell types can be analysed using recombinant adenovirus vectors.

TABLE 1
POSSIBLE CHIMERIC RECEPTOR COMBINATIONS

| | LIGAND | | TRANS | | CYTOSOLIC | | CYTOSOLIC | | CYTOSOL⁴ |
|---|---|---|---|---|---|---|---|---|---|
| | BINDING | SPACER | MEMBRANE | SPACER | COMPONENT | SPACER | COMPONENT | SPACER | SPACERS |
| A | TAA SCFV | G1 | TCR ZETA | OPT** | TCR ZETA | OPT | OPT | OPT | OPT |
| | TAA SCFV | h | CD28 | OPT | CD28 | OPT | OPT | OPT | OPT |
| B | TAA SCFV | CD8 | TCR ZETA | OPT | TCR ZETA | OPT | OPT | OPT | OPT |
| | TAA SCFV | h | CD28 | OPT | CD28 | OPT | OPT | OPT | OPT |
| C | TAA SCFV | G1 | TCR ZETA | OPT | TCR ZETA | OPT | OPT | OPT | OPT |
| | TAA SCFV | G1 | IL2 R β | OPT | IL2 R β | OPT | IL2 R γ | OPT | OPT |
| D | TAA SCFV | G1 | TCR ZETA | OPT | TCR ZETA | OPT | CD28 | OPT | OPT |
| E | TAA SCFV | h | TCR ZETA | OPT | TCR ZETA | OPT | CD28 | OPT | OPT |
| F | TAA SCFV | G1 | TCR ZETA | OPT | TCR ZETA | OPT | IL2 R β | OPT | IL2 R γ |

A, B and C describe pairs of genes coding for pairs of chimeric receptors
D, E and F describe fusion chimeric receptors, as shown in C one of a pair of receptors may be a fusion receptor
TAA SCFV denotes a single chain FV to a Tumour associated antigen
For a pair of chimeric receptors the SCFVs may bind the same or different epitopes of the same antigen or different antigens on the same or different cells.
G1 is the IgG CH₃CH₂ HINGE spacer construct described in the text
h denotes theIgG hinge plus part of the CD28 extracelluar component described in the text
⁴one or more further cytosolic and or spacer components
**OPT = optional

TABLE 2
CHIMERIC RECEPTOR CONSTRUCTS AND CELL LINE NOMENCLATURE

| | CONSTRUCTION | EXPRESSION | CELL |
|---|---|---|---|
| CONSTRUCT | PLASMID | PLASMID | LINES |
| hCTMO1 scFv/CD8/TCR zeta | pBS3 | pTB3 | TB3 |
| hP67.6 scFv/CD8/TCR zeta | pBS5 | pTB5 | TB5 |
| hCTMO1 scFv/CD8/CD28 | pHMF 320 | pHMF 332 | HGT 1 |
| hCTMO1 scFv/CD8/TCR zeta-CD28 | pHMF 326 | pHMF 334 | HGT 2 |
| hP67.6 scFv/G1/TCR zeta | pHMF 342 | pHMF 351 | HGT 6 & 16 |
| hP67.6 scFv/G1/TCR zeta-CD28 | pHMF 354 | pHMF 355 | HGT 7 & 17 |
| hP67.6 scFv/h/CD28 | pHMF 350 | pHMF 353 | HGT 8 & 14 |
| hP67.6 scFv/G1/CD28 | pHMF 375 | pHMF 376 | HGT 23 |
| hP67.6 scFv/G1/CD28-TCR zeta | pHMF 372 | pHMF 373 | HGT 21 |
| hP67.6 scFv/G1/CD28-TCR zeta-CD28 | pHMF 379 | pHMF 380 | HGT 26 |
| hP67.6 scFv/h.28/TCR zeta | pHMF 377 | pHMF 378 | HGT 24 |
| hP67.6 scFv/h.28/TCR zeta - CD28 | pHMF 363 | pHMF 364 | HGT 20 |
| hP67.6 scFv/h.28/CD28 - TCR zeta | pHMF 369 | pHMF 371 | HGT 22 |

G1 is the IgG hinge CH2 CH3 spacer
h is the IgG hinge component plus part of CD28 extracellular domain spacer.
h.28 is the IgG hinge component plus part of CD28 extracellular domain and amino acid residues L, D, P & K spacer.
Expression plasmids pTB3 and pTB5, pHMF 334, 351, 355, 378 and 364 include the TCR zeta transmembrane domain.
Expression plasmids pHMF 332, 353, 376, 373, 380 and 371 include the CD28 transmembrane domain.

1. A method of activating a cell as a result of one type of extracellular interaction between said first cell and a molecule associated with a second target cell characterised in that said first cell is provided with a DNA delivery system comprising DNA coding for one or more recombinant chimeric receptors comprising two or more different cytoplasmic signalling components, wherein said cytoplasmic components are not naturally linked, and at least one is derived from a membrane spanning polypeptide.

2. A method according to claim 1 wherein the cytoplasmic signalling components are capable of acting together cooperatively.

3. A method according to claim 1 or claim 2 wherein said DNA additionally codes for signal peptide, binding and/or transmembrane components of said one or more chimeric receptors, wherein the binding component is capable of recognising a cell surface molecule on a target cell.

4. A method according to claim 3 wherein the signal peptide, transmembrane and cytoplasmic signalling components and all or part of the binding component are coded for by a single DNA coding sequence.

5. A method according to claim 3 wherein each cytoplasmic signalling component is coded for by a separate DNA coding sequence, each of DNA sequence additionally coding for a signal peptide, a transmembrane component and all or part of a binding component.

6. A method according to claim 4 or claim 5 wherein said DNA codes for part of said binding component and an additional separate DNA coding sequence codes for the remainder of the binding component.

7. A method according to claim 5 or claim 6 wherein the binding component coded for by one DNA sequence is capable of participating in the same type of extracellular binding event as the binding component coded for by any other DNA sequence.

8. A method according to claim 7 wherein each binding component binds to the same molecule associated with the target cell.

9. A method according to claim 8 wherein each binding component is the same.

10. A method according to any one of claims 1 to 9 wherein the one or more recombinant chimeric receptors are capable of recognising a viral or cell surface molecule on a target cell.

11. A DNA delivery system comprising DNA in association with a carrier said DNA coding for a recombinant chimeric receptor capable of one type of extracellular interaction and comprising two or more different cytoplasmic signalling components which are not naturally linked, and wherein at least one of said cytoplasmic components is derived from a membrane spanning polypeptide.

12. A DNA delivery system comprising DNA in association with a carrier said DNA coding for two or more recombinant chimeric receptors each capable of the same one type of extracellular interaction and wherein each of said receptors comprises one or more different cytoplasmic signalling components which are not naturally linked, and wherein at least one of said cytoplasmic components is derived from a membrane spanning polypeptide.

13. A DNA delivery system according to claim 11 wherein said DNA codes in reading frame for:
i) a signal peptide component;
ii) a binding component capable of recognising a cell surface molecule on a target cell;
iii) a transmembrane component,
iv) two or more different cytoplasmic signalling components which are not naturally linked, and wherein at least one of said cytoplasmic components is derived from a membrane spanning polypeptide; and optionally
v) one or more spacer regions linking any two or more of said i) to iv) components.

14. A DNA delivery system according to claim 11 wherein said DNA comprises 1) a first DNA which codes in reading frame for:
i) a signal peptide component;
ii) part of a binding component;
iii) a transmembrane component;
iv) two or more cytoplasmic signalling components which are not naturally linked, and wherein at least one of said cytoplasmic components is derived from a membrane spanning polypeptide; and optionally
v) one or more spacer regions linking any two or more of said i) to iv) components; and 2) a second separate DNA which codes in reading frame for a signal peptide component and a further part of the binding component ii) coded for by said first DNA, such that the binding component parts together are capable of recognising a cell surface molecule on a target cell.

15. A DNA delivery system according to claim 12 wherein said DNA comprises a first and a second separate DNA each of which codes in reading frame for:
i) a signal peptide component;
ii) a binding component capable of recognising a cell surface molecule on a target cell;
iii) a transmembrane component;
iv) one or more different cytoplasmic signalling components which are not naturally linked, and wherein at least one of said cytoplasmic components is derived from a membrane spanning polypeptide; and optionally
v) one or more spacer regions linking any two or more of said i) to iv) components; provided that said first DNA codes for at least one signalling component iv) that is not coded for by said second DNA.

16. A DNA delivery system according to claim 12 wherein said DNA comprises 1) a first and a second separate DNA each of which codes in reading frame for:
i) a signal peptide component;
ii) one part of a binding component;
iii) a transmembrane component;
iv) one or more different cytoplasmic signalling components which are not naturally linked, and wherein at least one of said cytoplasmic components is derived from a membrane spanning polypeptide; and optionally
v) one or more spacer regions linking any two or more of said i) to iv) components; provided that said first DNA codes for at least one signalling component iv) that is not coded for by said second DNA; and 2) a separate third and fourth DNA each of which codes in reading frame for a signal peptide component and a further part of the binding component ii) coded for by said first and second DNA respectively, such that the binding component parts together provided by the first and third DNA and together provided by the second and fourth DNA are each capable of recognising a cell surface molecule on a target cell.

17. A DNA delivery system according to claims 13 to 16 wherein each signal peptide component is an immunoglobulin signal sequence.

18. A DNA delivery system according to claims 15 to 17 wherein the binding component coded for by said first DNA is the same as the binding component coded for by said second DNA.

19. A DNA delivery system according to claims 13 to 18 wherein the binding component is an antibody or an antigen binding fragment thereof.

20. A DNA delivery system according to claim 19 wherein the antibody or fragment thereof is an engineered human antibody or antigen binding fragment thereof.

21. A DNA delivery system according to claims 18 to 20 wherein the binding component is a single chain Fv fragment.

22. A DNA delivery system according to claims 18 to 20 wherein the binding component is a Fab′ fragment.

23. A DNA delivery system according to any one of claims 13 to 22 wherein the transmembrane component is derived from all or part of the alpha, beta or zeta chain of the T-cell receptor, CD28, CD8, CD4, a cytokine receptor or a colony stimulating factor receptor.

24. A DNA delivery system according to claim 23 wherein the transmembrane component is derived from all or part of CD28.

25. A DNA delivery system according to any one of claims 11 to 24 wherein the cytoplasmic signalling components are capable of acting together cooperatively.

26. A DNA delivery system according to any one of claims 13 to 25 wherein the cytoplasmic signalling components are derived from all or part of the cytoplasmic domains of a zeta, eta or epsilon chain of the T-cell receptor, CD28, the γ chain of a Fc receptor, a cytokine receptor, a colony stimulating factor receptor, a tyrosine kinase or an adhesion molecule, B29, MB-1, CD3 delta, CD3 gamma, CD5 or CD2.

27. A DNA delivery system according to claim 26 wherein the cytoplasmic signalling components are ITAM containing cytoplasmic components.

28. A DNA delivery system according to claim 26 or claim 27 wherein the cytoplasmic signalling components are derived from all or part of CD28 and/or the zeta chain of the T-cell receptor.

29. A DNA delivery system according to any one of claims 11 to 28 wherein the cytoplasmic signalling components are in any orientation relative to one another.

30. A DNA delivery system according to any one of claims 13 to 29 wherein said DNA coding for components i) to iv) additionally codes for one or more spacer regions linking the binding component ii) and the transmembrane component iii).

31. A DNA delivery system according to claim 30 wherein two or more different spacer regions link the binding component ii) and the transmembrane component iii), both regions either being coded for by one DNA sequence or when a first and second DNA sequence is present one region being coded for by said first DNA and the other different region being coded for by said second DNA.

32. A DNA delivery system according to claims 30 or claim 31 wherein the spacer region is selected to provide one or more free thiol groups.

33. A DNA delivery system according to claims 30 to 32 wherein the spacer region is derived from all or part of the extracellular region of CD8, CD4 or CD28.

34. A DNA delivery system according to claims 30 or claim 32 wherein the spacer region is all or part of an antibody constant region.

35. A DNA delivery system according to claims 30 to 32 wherein the spacer region is derived from all or part of an antibody hinge region linked to all or part of the extracellular region of CD28.

36. A DNA delivery system according to any one of claims 11 to 35 wherein the carrier is a viral vector or a non-viral vector.

37. A DNA delivery system according to claim 36 wherein the non-viral vector is a liposomal vector.

38. A DNA delivery system according to claim 37 wherein the carrier is a targeted non-viral vector.

39. A DNA delivery system according to claim 38 wherein the targeted vector is an antibody targeted liposome.

40. A DNA delivery system according to claim 38 wherein the targeted vector is an antibody targeted condensed DNA.

41. A DNA delivery system according to claim 40 wherein the targeted vector is an antibody targeted protamine or polylysine condensed DNA.

42. A DNA delivery system according to claim 38 wherein the targeted vector is antibody targeted naked DNA.

43. A DNA delivery system according to claims 39 to 42 wherein the antibody is a whole antibody or an antigen binding fragment thereof.

44. A DNA delivery system according to claim 43 wherein the antibody is an engineered human antibody or an antigen binding fragment thereof.

45. An effector cell transfected with a DNA delivery system according to any one of claims 1 to 444.

46. An effector cell according to claim 45 which is a lymphocyte, a dendritic cell, a B-cell, a haematopoietic stem cell, a macrophage, a monocyte or a NK cell.

47. An effector cell according to claim 46 which is a cytotoxic T-lymphocyte.

48. A DNA delivery system according to any one of claims 11 to 47 for use in the treatment of infectious disease, inflammatory disease. cancer, allergic/atopic disease, congenital disease, dermatologic disease, neurologic disease, transplants and metabolic/idiopathic disease.

49. A DNA delivery system according to claim 48 for use in the treatment of rheumatoid arthritis, osteoarthritis, inflammatory bowel disease, asthma, eczema, cystic fibrosis, sickle cell anaemia, psoriasis, multiple sclerosis, organ or tissue transplant rejection, graft-versus-host disease or diabetes.

50. A pharmaceutical composition comprising a DNA delivery system according to any one of claims 11 to 44 together with one or more formulatory agents.

51. A pharmaceutical composition according to claim 50 wherein the formulatory agent is a suspending, preservative, stabilising and/or dispersing agent.

52. DNA coding for a recombinant chimeric receptor for use in a delivery system according to any one of claims 11 to 44.
